(19) United States
(12) Patent Application Publication
(10) Pub. No.: US 2004/0219574 A1
(43) Pub. Date: Nov. 4, 2004

(54) Method for the specific fast detection of bacteria which are harmful to beer

(76) Inventors: Claudia Beimfohr, Munchen (DE); Jiri Snaidr, Vierkirchen (DE)

Correspondence Address: KNOBBE MARTENS OLSON & BEAR LLP, 2040 MAIN STREET, FOURTEENTH FLOOR, IRVINE, CA, 92614, US

(21) Appl. No.: 10/740,926

(22) Filed: Dec. 18, 2003

Related U.S. Application Data
(63) Continuation of application No. PCT/EP02/06808, filed on Jun. 19, 2002.

(30) Foreign Application Priority Data
Jun. 19, 2001 (DE) 10129 410.7

(51) Int. Cl. C12Q 1/68; C07H 21/04

(52) U.S. Cl. 435/6; 536/23.7

(57) ABSTRACT

The invention relates to a method for the specific fast detection of bacteria which is harmful to beer by in situ hybridization. The invention also relates to oligonucleotide probes for use with said method and kits enabling the inventive detection method to be carried out.

### CROSS REFERENCE TO RELATED APPLICATION

The present application is a continuation of PCT application Serial No. PCT/EP02/06808, filed Jun. 19, 2002, entitled “METHOD FOR THE SPECIFIC FAST DETECTION OF BACTERIA WHICH IS HARMFUL TO BEER,” the disclosure of which is incorporated herein by reference in its entirety; which claims priority from German Patent Application Serial No. 101 29 410.7, filed Jun. 19, 2001, the disclosure of which is incorporated herein by reference in its entirety.

### BACKGROUND OF THE INVENTION

1. Field of the Invention

The invention relates to a method for the specific fast detection of bacteria, which are harmful to beer by in situ hybridization. The invention also relates to oligonucleotide probes suitable for use with said method and kits enabling the inventive detection method to be carried out.

2. Description of the Related Art

Annual beer production in the EU amounts to approximately 313 million hectoliters, of which 112 million hectoliters are produced in Germany. With approximately 1270 resident breweries and a yearly turnover of approximately DM 18 billion, the brewing process is one of the most important industrially used biotechnological processes in Germany (“Data from the Brewery Trade Europe 1999”, Deutscher Brauer Bund, 2001, Bonn, http://www.brauer-bund.de).

In order to meet the high quality standards for the beer product, besides the choice of raw materials and the brewing process itself, microbiological quality control is also very important.

Due to the highly selective and partly bactericidal effect of beer, a very narrow spectrum of microorganisms has colonized in brewery plants. In order to persist in the beer environment, the microorganisms must tolerate a low pH, an anaerobic atmosphere, hop bitters, alcohol and a very low content and variety of nutrients and growth substances (W. Back, Farbatlas und Handbuch der Getränkebiologie, 1994, Verlag Hans Carl, Nümberg). Microorganisms which are harmful to beer are thus predominantly lactic-acid bacteria of the genera Lactobacillus and Pediococcus as well as members of the genera Pectinatus and Megasphaera.

The term lactic-acid bacteria includes all Gram-positive, non-spore-forming catalase negative rods and cocci. Up to now nine different genera (Lactobacillus, Lactococcus, Leuconostoc, Carnobacterium, Bifidobacterium, Enterococcus, Pediococcus, Weissella and Streptococcus) are subsumed under the term lactic-acid bacteria. In phylogenetic terms, all members of lactic-acid bacteria are classified as belonging to the class of Gram-positive bacteria with a low GC content in the DNA (Brock, Mikrobiologie, 2001, Spektrum Akademischer Verlag GmbH, Heidelberg-Berlin).

Some of these genera are very important for the food industry. Thus, members of the genera Lactobacillus, Lactococcus and Streptococcus are used for the fermentation of cheese, yogurt, buttermilk, sour cream, “sauerkraut”, meat and sausage products and other foodstuffs.

However, lactic-acid bacteria are by no means always relevant in a positive meaning in the food and beverage industry. In fact, some members of various genera play an important role in causing foods to spoil.

For example, some species of the genera Lactobacillus and Pediococcus are responsible for more than 90% of beer spoilage caused by microbial growth. The spoilage of product caused by these organisms is accompanied by changes in taste and smell and usually by the beer becoming very cloudy.

Members of the very heterogenous genus Lactobacillus are described as Gram-positive, non-spore-forming, homo- or heterofermentative, catalase negative and usually non-motile rods. Presently about 50 different species are included in this genus, of which only a very small number are demonstrably harmful to beer.

Pediococci are characterized as Gram-positive, non-spore-forming, homofermentative, catalase negative cocci which mainly occur in tetrads. Also in this genus only a few species are able to grow in beer and are thus capable of making it go off (Brock, Mikrobiologie, 2001, Spektrum Akademischer Verlag GmbH Heidelberg-Berlin; Allgemeine Mikrobiologie, H. Schlegel, 1992, Georg Thieme Verlag, Stuttgart).

The following species among the group of lactic-acid bacteria are described as being potentially harmful to beer: Lactobacillus brevis, Lactobacillus buchneri, Lactobacillus lindneri, Lactobacillus coryniformis, Lactobacillus plantarum, Lactobacillus pseudoplantarum, Lactobacillus casei, Lactobacillus paracasei, Lactobacillus fructivorans, Lactobacillus perolens, Lactobacillus rhamnosus, Lactobacillus frigidus, Pediococcus damnosus, Pediococcus inopinatus (W. Back, Farbatlas und Handbuch der Getränkebiologie, 1994, Verlag Hans Carl, Nümberg). Important in practice are especially Lactobacillus brevis, Lactobacillus lindneri and Pediococcus damnosus.

In addition to the Gram-positive lactic-acid bacteria mentioned, also some Gram-negative bacteria of the genera Pectinatus and Megasphaera are known to spoil beer. The rod-shaped cells of the genus Pectinatus are strictly anaerobic, motile and slightly curved bacteria. The spherical or slightly oval coccoid cells of the genus Megasphaera also count among the strictly anaerobic microorganisms (W. Back, Farbatlas und Handbuch der Getränkebiologie, 1994, Verlag Hans Carl, Nümberg). While the aforementioned Gram-positive beer contaminants are present in the beer as so-called primary contaminants, contamination with Megasphaera and Pectinatus mostly occur at the bottling station directly at the bottler, which is why the bacteria are also called secondary contaminants.

Pectinatus frisingensis, Pectinatus cerevisiiphilus, Megasphaera cerevisiae are Gram-negative bacteria, which have up to now been described as being harmful for beer (W. Back, Farbatlas und Handbuch der Getränkebiologie, 1994, Verlag Hans Carl, Nümberg).

The heterogeneity of microorganisms, which are harmful to beer, places the highest demands on microbiological quality control in breweries. Add to this in comparison to the batch size of up to 1000 hectoliters the volume of the sample used for analysis is very small (as a rule 250 to 500 ml) (Back, W. und Pöschl, P., Bypass-Membranfiltration [BM-System]—Verbesserung des Spurennachweises nach der Filtration, Brauwelt 138:2312-2315).

Up to now the standard way of detecting bacteria which are harmful to beer has been cultivation. Several selective media are available, such as NBB, VLB-S7S, UBA and MRS. All selective media used are intended to promote the growth of the harmful beer bacteria, whereas at the same time the growth of microbial passenger flora or brewery-specific yeast cultures are blocked by inhibitors. All culture methods have in common the fact that they only allow a qualitative statement about the presence or absence of beer contaminants. A quantitative test is not carried out. Traditional culture methods are very time-consuming, taking up to twelve days for the cultivation. This leads to high indirect costs due to the need to store the beer until the quality control has been completed and the production batch has been approved for release.

If the result of the cultivation process is positive, a subsequent characterization of the detected beer contaminant would be useful. Such a broader determination has not been performed up to now, because user-friendly methods have not been available for this purpose. For an exact identification of the bacterium, which is harmful to beer, further physiological tests (such as Gram's stain, sugar utilization series) would have to be carried out. On the one hand this is very time-consuming and on the other hand the proper performance of these analyses calls for a high level of qualification on the part of the staff performing such analyses. But dispensing with a more accurate analysis also means dispensing with the advantages inherent in such an analysis. An exact knowledge of the contaminant permits conclusions to be drawn about the possible contamination source and thus provides an opportunity to counteract further contaminations by effectively combating the beer contaminant. In this context it is also helpful to know whether the beer contaminant is always the same, or whether different germs are responsible for the product spoilage.

As a logical consequence of the difficulties arising from traditional cultivation methods in the detection of bacteria which are harmful for beer, alternative detection methods based on nucleic acids therefore would be useful.

### SUMMARY OF THE INVENTION

Some embodiments relate to isolated oligonucleotides. The isolated oligonucleotides can include oligonucleotides having any of the sequences of SEQ ID NOs 1-442.

Other embodiments relate to methods for detecting bacteria in a sample, including those which are harmful to beer. The methods can include the steps of cultivating the bacteria contained in the sample; fixing the bacteria contained in the sample; incubating the fixed cells with at least one oligonucleotide having a sequence of any of SEQ ID NOs. 1-442, in order to achieve hybridization; removing or washing off the non-hybridized oligonucleotides; and detecting the hybridized oligonucleotides and thereby the bacteria, including those which are harmful to beer. The sample in any of the methods can be, for example, a beer sample, a yeast sample, a rinse water sample, or a food sample. The bacteria which are harmful to beer can be, for example, lactic-acid bacteria or Gram-negative bacteria. The lactic-acid bacteria can belong to the genera of Lactobacillus or Pediococcus, and preferably from the species Pediococcus damnosus, Lactobacillus coryniformis, Lactobacillus perolens, Lactobacillus buchneri, Lactobacillus plantarum, Lactobacillus fructivorans, Lactobacillus lindneri, Lactobacillus casei, Lactobacillus brevis. The Gram-negative bacteria can belong to the genera Pectinatus and Megasphaera, preferably from the species Pectinatus frisingensis, Pectinatus cerevisiiphilus and Megasphaera cerevisiae. The methods can further include quantifying and visualizing the bacteria with hybridized oligonucleotides, including those bacteria which are harmful to beer.

Still further embodiments relate to methods for the detection of bacteria which are harmful to beer in a sample using an oligonucleotide with any of sequence from the sequences of SEQ ID NOs. 1-442. The bacteria which is harmful to beer can be, for example, a lactic-acid bacteria or a Gram-negative bacteria, and preferably the lactic-acid bacteria or the Gram-negative bacteria can be Lactobacillus, Pediococcus, Pectinatus or Megasphaera. Preferably the Lactobacillus, Pediococcus, Pectinatus or Megasphaera can be, for example Pediococcus damnosus, Lactobacillus coryniformis, Lactobacillus perolens, Lactobacillus buchneri, Lactobacillus plantarum, Lactobacillus fructivorans, Lactobacillus lindneri, Lactobacillus casei, Lactobacillus brevis, Pectinatus frisingensis, Pectinatus cerevisiiphilus or Megasphaera cerevisiae.

Also, some embodiments relate to kits for performing any of the described methods. The kits can include, for example, at least one oligonucleotide having the sequence of SEQ ID NOs. 1-442. The kits also can include, for example, at least one oligonucleotide in a hybridization solution, a washing solution, one or more fixation solutions, or a cell breaking solution or enzyme solution.

### DETAILED DESCRIPTION OF THE PREFERRED EMBODIMENT

In PCR, the polymerase chain reaction, the sample under study is enriched (mostly in NBB medium) prior to amplification of a characteristic segment of the particular bacterial genome with specific primers. If the primer finds its target site, millions of amplicons of a segment of the genetic information are generated. In the subsequent analysis, using for instance an agarose gel in order to separate DNA fragments, a qualitative evaluation can be made. In the simplest case, this results in the information that the target sites for the primers used are present in the analyzed sample. Other conclusions are not possible, since the target sites may be derived from a living bacterium, a dead bacterium or a naked DNA. Differentiation is not possible here, which is very problematic for the analysis of beer samples. The beer and various selective media based on wort contain a large number of dead lactic-acid bacteria which are used for biological acidification. As the PCR reaction is also positive in the presence of such dead bacteria, or their naked DNA, false positive results are almost inevitable here. On the other hand various substances present in the beer may cause inhibition of the DNA amplifying enzyme, the Taq polymerase. This is a frequent cause of false negative results. A further development of the PCR technique is quantitative PCR, in which an attempt is made to create a correlation between the amount of bacteria present and the amount of DNA obtained and amplified. Advantages of the PCR are its high specificity, ease of application and low expenditure of time. Significant disadvantages are its high susceptibility to contaminations with consequent false positive results as well as the aforementioned lack of possibility of distinguishing between living and dead cells or naked DNA, respectively.

A unique approach for realizing the specificity of the molecular biological methods such as PCR without accepting the disadvantages involved in said method is the method of fluorescent in situ hybridization (FISH; Amann2, R. I., W. Ludwig and K. -H. Schleifer, Phylogenetic identification and in situ detection of individual microbial cells without cultivation, Microbiol. Rev. (1995) 59:143-169). Using this method bacterial species, genera or groups can be visualized and identified highly specifically.

The FISH technique is based on the fact that there are certain molecules present in bacterial cells, which due to their vital function have been mutated to only a small degree in the course of evolution. These are the 16S and the 23S ribosomal ribonucleic acids (rRNA). Both are constituents of the ribosomes, the sites of protein biosynthesis and can serve as specific markers due to their ubiquitous distribution, their size and their structural and functional constancy (Woese, C. R., Bacterial evolution, Microbiol. Rev. (1987) 51: 221-271). Using comparative sequence analysis, phylogenetic relationships can be derived solely from these data. For this, these sequence data have to be aligned. In an alignment, which is based on knowledge of the secondary and tertiary structures of these macromolecules, the homologous positions of the ribosomal nucleic acids are correlated.

Based on these data, phylogenetic calculations can be performed. By using state of the art computer technology it is possible to make even large-scale calculations fast and efficiently, as well to create large databases containing the aligned sequences of 16S rRNA and 23S RNA. Through fast access to this data material, newly obtained sequences can be analyzed phylogenetically in a short period of time. These rRNA databases can be used to construct species-specific or genus-specific gene probes. Hereby, all available rRNA sequences are compared and probes are designed for certain sequence regions, which specifically detect a bacterial species, genus or group.

In FISH (fluorescence in situ hybridization) these gene probes, which are complementary to a certain region on the ribosomal target sequence, are introduced into the cell. Usually, the gene probes are small, 16-20 bases long, single-stranded deoxyribonucleic acid fragments, and are directed to a target region, which is typical for a bacterial species or a bacterial group. If the fluorescence-labeled gene probe finds its target sequence in a bacterial cell, it binds to it and the cell can be detected due to the fluorescence in the fluorescence microscope.

Generally, the FISH analysis is performed on a microscope slide, because the bacteria are visualized, i.e. are made visible, by irradiation with high energetic light during evaluation. But this is exactly where one of the disadvantages of the classical FISH analysis resides. As only relatively small volumes can be analyzed on a microscope slide by its very nature, the sensitivity of the method can be unsatisfactory and not adequate for a reliable analysis. The present invention thus combines the advantages of the classical FISH analysis with those of cultivation. A comparably short cultivation step ensures that the bacteria to be detected are present in a sufficient amount before detection of the bacteria is performed by specific FISH.

The performance of the method of the present invention for specific fast detection of bacteria, which are harmful to beer, comprises the following steps:

cultivating the bacteria present in the sample under study

fixing the bacteria present in the sample

incubating the fixed bacteria with nucleic acid probe molecules in order to achieve hybridization

removing or washing off the non-hybridized nucleic acid probe molecules, and

detecting the bacteria hybridized with the nucleic acid probe molecules.

Within the scope of the present invention “cultivating” means propagating the bacteria contained in the sample in a suitable culture medium. Methods suitable for this purpose are well known to the skilled artisan.

Within the scope of the present invention “fixing” of the bacteria means a treatment with which the cell envelope of the bacteria is made permeable for nucleic acid probes. Ethanol is usually used for fixation. If the cell wall cannot be penetrated by the nucleic acid probes using these techniques, the person of skill in the art will know sufficient further techniques which lead to the same result. These include, for instance, methanol, mixtures of alcohols, a low percentage of paraformaldehyde solution or a diluted formaldehyde solution, enzymatic treatments, or similar. In an especially preferred embodiment of the method of the present invention an enzymatic step may be followed in order to cause complete disintegration of the bacteria. Enzymes, which can be used for this step are for instance lysozyme, proteinase K and mutanolysine. One of ordinary skill in the art will know sufficient further techniques and will easily find out which agent is useful for cell disintegration, depending on which bacteria is involved.

Within the scope of the present invention the fixed bacteria are incubated for the “hybridization” using fluorescence-labeled nucleic acid probes. These nucleic acid probes, consisting of an oligonucleotide and a marker linked thereto, are then able to penetrate the cell envelope in order to bind to the target sequence corresponding to the nucleic acid probe within the cell. The binding is to be understood as a formation of hydrogen bonds among complementary nucleic acid regions.

The nucleic acid probe may hereby be complementary to a chromosomal or episomal DNA, but also to an mRNA or rRNA of the microorganisms to be detected. It is advantageous to select a nucleic acid probe that is complementary to a region present in copies of more than 1 in the microorganism to be identified. The sequence to be detected is preferably present in 500-100,000 copies per cell, especially preferred in 1,000-50,000 copies. For this reason, the rRNA is used preferably as a target site, since in each active cell the ribosomes as sites of protein biosynthesis are present in many thousand copies.

The nucleic acid probe within the meaning of the invention may be a DNA or RNA probe comprising usually between 12 and 1,000 nucleotides, preferably between 12 and 500, more preferably between 12 and 200, especially preferably between 17 and 50 and between 15 and 40, and most preferably between 17 and 25 nucleotides. The selection of the nucleic acid probes is done according to the criteria of whether a complementary sequence is present in the microorganism to be detected. By selecting a defined sequence, a bacterial species, a bacterial genus or an entire bacterial group may be detected. In a probe consisting of 15 nucleotides, 100% of the sequence should be complementary. In oligonucleotides consisting of more than 15 nucleotides, one or more, especially one, two or three mismatches are allowed.

In the context of the method of the present invention, the inventive nucleic probe molecules comprise the lengths and sequences as set out below (all nucleic probe molecules are noted in 5′ to 3′ direction).

The nucleic acid probe molecules of the present invention are useful for the detection of lactic-acid bacteria which are harmful to beer belonging to the genera Lactobacillus and Pediococcus, especially belonging to the species Pediococcus damnosus, Lactobacillus coryniformis, Lactobacillus perolens, Lactobacillus buchneri, Lactobacillus plantarum, Lactobacillus fructivorans, Lactobacillus lindneri, Lactobacillus casei, Lactobacillus brevis as well as for the detection of Gram-negative bacteria, which are harmful to beer, belonging to the genera of Pectinatus and Megasphaera, especially to the species Pectinatus frisingensis, Pectinatus cerevisiiphilus, Megasphaera cerevisiae and are used correspondingly in the detection method according to the invention.

| | |
|---|---|
| TGG TGA TGC AAG CAC CAC | SEQ ID No. 1 |
| ATG MTG ATG CAA GCA CCA R | SEQ ID No. 2 |
| CAT GCG GTC TCC GTG GTT | SEQ ID NO. 3 |

The sequences SEQ ID Nos. 1 to 3 are especially useful for the detection of Lactobacillus perolens. SEQ ID Nos. 1 to 3 are preferred embodiments of the invention.

| | |
|---|---|
| ACG CTG AGT GGC GCG GGT | SEQ ID No. 4 |

SEQ ID No. 4 is especially useful for the detection of Lactobacillus buchneri. SEQ ID No. 4 is a preferred embodiment of the invention.

| | |
|---|---|
| GCG GGA CCA TCC AAA AGT G | SEQ ID No. 5 |

SEQ ID No. 5 is especially useful for the detection of Lactobacillus plantarum. SEQ ID No. 5 is a preferred embodiment of the invention.

| | |
|---|---|
| GGC GGC AGG GTC CAA AAG | SEQ ID No. 6 |

SEQ ID No. 6 is especially useful for the detection of Lactobacillus fructivorans. SEQ ID No. 6 is a preferred embodiment of the invention.

| | |
|---|---|
| CGT CAC GCC GAC AAC AGT | SEQ ID No. 7 |

SEQ ID No. 7 is especially useful for the detection of Lactobacillus casei. SEQ ID No. 7 is a preferred embodiment of the invention.

| | |
|---|---|
| GGC GGC TAG TTC CCT AAA | SEQ ID No. 8 |

SEQ ID No. 8 is especially useful for the detection of Lactobacillus coryniformis. SEQ ID No. 8 is a preferred embodiment of the invention.

| | |
|---|---|
| ACC GTC AAC CCT TGA ACA GT | SEQ ID No. 9 |
| GAC TCC CGA AGG TTA TCT | SEQ ID No. 10 |

SEQ ID Nos. 9 and 10 are preferred embodiments of the invention. These sequences are especially useful for the detection of Lactobacillus brevis.

| | |
|---|---|
| TCG GTC AGA TCT ATC GTC | SEQ ID No. 11 |

SEQ ID No. 11 is especially useful for the detection of Lactobacillus lindneri. SEQ ID No. 11 is a preferred embodiment of the invention.

| | |
|---|---|
| GCT ACG TAT CAC AGC CTT | SEQ ID No. 12 |

SEQ ID No. 12 is especially useful for the detection of Pediococcus damnosus. SEQ ID No. 12 is a preferred embodiment of the invention.

| | |
|---|---|
| GCG GCG GAC TCC GTA AAG | SEQ ID No. 13 |

SEQ ID No. 13 is especially useful for the detection of Lactobacillus lindneri.

| | |
|---|---|
| GCT ACC CAY GCT TTC GAG | SEQ ID No. 14 |

SEQ ID No. 14 is useful for the detection of the genera Pediococcus and Lactobacillus.

| | |
|---|---|
| CCA ATG CAC TTC TTC GGT | SEQ ID No. 15 |

SEQ ID No. 15 is especially useful for the detection of bacteria belonging to the genus of Pediococcus, especially P. acidilactici, P. pentosaceus, P. damnosus and P. parvulus.

| | |
|---|---|
| GCT CGC TCC CTA AAA GGC | SEQ ID No. 16 |

SEQ ID No. 16 is useful for the detection of L. casei.

| | |
|---|---|
| ACT GCA AGC AGC TTC GGT | SEQ ID No. 17 |

SEQ ID No. 17 is especially useful for the detection of L. coryniformis.

| | |
|---|---|
| CGC CGC GGA TCC ATC CAA | SEQ ID No. 18 |

SEQ ID No. 18 is especially useful for the detection of L. fructivorans.

| | |
|---|---|
| TGC TTT CGA GAC CTC AGC | SEQ ID No. 19 |
| TTA CAA GAC CAG ACA GCC | SEQ ID No. 20 |

SEQ ID Nos. 19 and 20 are useful for the detection of P. damnosus.

| | |
|---|---|
| ACC GTC AAC CCT TGA ACA GT | SEQ ID No. 21 |

SEQ ID No. 21 is useful for the detection of L. brevis.

| | |
|---|---|
| ACG CCG CGG GAC CAT CCA | SEQ ID No. 22 |
| AGT TCG CCA CTC ACT CAA | SEQ ID No. 23 |

SEQ ID Nos. 22 and 23 are useful for the detection of L. plantarum.

| | |
|---|---|
| CGC TAC CCA TGC TTT CGK G | SEQ ID No. 24 |
| CCA CTA CCC ATG CTT TCG AG | SEQ ID No. 25 |

SEQ ID Nos. 24 and 25 are useful for the detection of the genera of Pediococcus and Lactobacillus.

| | |
|---|---|
| CAA GCA CCA GCT ATC AGT | SEQ ID No. 26 |

SEQ ID No. 26 is useful for the detection of L. lindneri.

| | |
|---|---|
| ACG TCA TTC AAC GGA AGC | SEQ ID No. 27 |

SEQ ID No. 27 is useful for the detection of L. brevis.

| | |
|---|---|
| AGC TTC GAT GCA AGC ATC | SEQ ID No. 28 |
| TACAAGACCAGACAGCCG | SEQ ID No. 29 |
| GTTACAAGACCAGACAGC | SEQ ID No. 30 |
| ACAAGACCAGACAGCCGC | SEQ ID No. 31 |
| CGTCAGTTACAAGACCAG | SEQ ID No. 32 |
| GCGTCAGTTACAAGACCA | SEQ ID No. 33 |
| GAGACCTCAGCGTCAGTT | SEQ ID No. 34 |
| AGCGTCAGTTACAAGACC | SEQ ID No. 35 |
| CAAGACCAGACAGCCGCC | SEQ ID No. 36 |
| ACCCATGCTTTCGAGACC | SEQ ID No. 37 |
| ACGTATTACCGCGGCTCG | SEQ ID No. 38 |
| TAAAAAAACCGCCTGCGC | SEQ ID No. 39 |
| ATGCTTTCGAGACCTCAG | SEQ ID No. 40 |
| CCATGCTTTCGAGACCTC | SEQ ID No. 41 |
| TGCTTTCGAGACCTCAGC | SEQ ID No. 42 |
| CATGCTTTCGAGACCTCA | SEQ ID No. 43 |
| CCCATGCTTTCGAGACCT | SEQ ID No. 44 |
| AGACCTCAGCGTCAGTTA | SEQ ID No. 45 |
| CTTTCGAGACCTCAGCGT | SEQ ID No. 46 |
| CGAGACCTCAGCGTCAGT | SEQ ID No. 47 |
| GCTTTCGAGACCTCAGCG | SEQ ID No. 48 |
| TCGAGACCTCAGCGTCAG | SEQ ID No. 49 |
| TTTCGAGACCTCAGCGTC | SEQ ID No. 50 |
| TTCGAGACCTCAGCGTCA | SEQ ID No. 51 |
| TACGTATTACCGCGGCTC | SEQ ID No. 52 |
| AAAAAAACCGCCTGCGCT | SEQ ID No. 53 |
| GCTTCGATGCAAGCATCT | SEQ ID No. 54 |
| CAGCTTCGATGCAAGCAT | SEQ ID No. 55 |
| ATCAGCTTCGATGCAAGC | SEQ ID No. 56 |
| TCAGCTTCGATGCAAGCA | SEQ ID No. 57 |
| CAGCGTCAGTTACAAGAC | SEQ ID No. 58 |
| AAGACCAGACAGCCGCCT | SEQ ID No. 59 |
| TACCCATGCTTTCGAGAC | SEQ ID No. 60 |
| TAGCTCCCGAAGGTTACT | SEQ ID No. 61 |
| CGAAGGTTACTCCACCGG | SEQ ID No. 62 |
| CCGAAGGTTACTCCACCG | SEQ ID No. 63 |
| GCTCCCGAAGGTTACTCC | SEQ ID No. 64 |
| CCCGAAGGTTACTCCACC | SEQ ID No. 65 |
| TCCCGAAGGTTACTCCAC | SEQ ID No. 66 |
| CTCCCGAAGGTTACTCCA | SEQ ID No. 67 |

The SEQ ID Nos. 28 to 67 are especially useful for the detection of P. damnosus.

| | |
|---|---|
| CCGTCAACCCTTGAACAG | SEQ ID No. 68 |
| CATTCAACGGAAGCTCGT | SEQ ID No. 69 |
| ACCGTCAACCCTTGAACA | SEQ ID No. 70 |
| CTTAGCCTCACGACTTCG | SEQ ID No. 71 |
| TACCGTCAACCCTTGAAC | SEQ ID No. 72 |
| AACGGAAGCTCGTTCGAC | SEQ ID No. 73 |
| TTAGCCTCACGACTTCGC | SEQ ID No. 74 |
| GCAAGCACGTCATTCAAC | SEQ ID No. 75 |
| TCGCCACTCGCTTCATTG | SEQ ID No. 76 |
| TCAACGGAAGCTCGTTCG | SEQ ID No. 77 |
| TTCAACGGAAGCTCGTTC | SEQ ID No. 78 |
| CAAGCACGTCATTCAACG | SEQ ID No. 79 |
| CACGTCATTCAACGGAAG | SEQ ID No. 80 |
| TCATTCAACGGAAGCTCG | SEQ ID No. 81 |
| TGACTCCCGAAGGTTATC | SEQ ID No. 82 |
| CGTCATTCAACGGAAGCT | SEQ ID No. 83 |
| GCTTAGCCTCACGACTTC | SEQ ID No. 84 |
| TTCGCCACTCGCTTCATT | SEQ ID No. 85 |
| GTCATTCAACGGAAGCTC | SEQ ID No. 86 |
| CCTGCTTCTGGGCAGATT | SEQ ID No. 87 |
| CTGCTTCTGGGCAGATTT | SEQ ID No. 88 |
| GCACGTCATTCAACGGAA | SEQ ID No. 89 |
| CAACGGAAGCTCGTTCGA | SEQ ID No. 90 |
| ACGGAAGCTCGTTCGACT | SEQ ID No. 91 |
| AGCACGTCATTCAACGGA | SEQ ID No. 92 |
| TCTGGGCAGATTTCCCAC | SEQ ID No. 93 |
| CGGAAGCTCGTTCGACTT | SEQ ID No. 94 |
| AAGCACGTCATTCAACGG | SEQ ID No. 95 |
| GTTCGCCACTCGCTTCAT | SEQ ID No. 96 |
| CCCTGCTTCTGGGCAGAT | SEQ ID No. 97 |
| CTGACTCCCGAAGGTTAT | SEQ ID No. 98 |
| TGCTTCTGGGCAGATTTC | SEQ ID No. 99 |
| TTCTGGGCAGATTTCCCA | SEQ ID No. 100 |
| ACTCCCGAAGGTTATCTC | SEQ ID No. 101 |
| CTTCTGGGCAGATTTCCC | SEQ ID No. 102 |
| CTGGGCAGATTTCCCACG | SEQ ID No. 103 |
| ACTAATACGCCGCGGGAT | SEQ ID No. 104 |
| GTGCAAGCACGTCATTCA | SEQ ID No. 105 |
| ACGGCTGACTCCCGAAGG | SEQ ID No. 106 |
| TTAGACGGCTGACTCCCG | SEQ ID No. 107 |

The sequences SEQ ID Nos. 68 to 107 are useful for the detection of L. brevis.

| | |
|---|---|
| GTCACACCGTGAGCAGTT | SEQ ID No. 108 |
| CGTCACACCGTGAGCAGT | SEQ ID No. 109 |
| CCACTCGGTCAGATCTAT | SEQ ID No. 110 |
| GATGCAAGCACCAGCTAT | SEQ ID No. 111 |
| TCGGTCAGATCTATCGTC | SEQ ID No. 112 |
| CGGTCAGATCTATCGTCA | SEQ ID No. 113 |
| CTCGGTCAGATCTATCGT | SEQ ID No. 114 |
| TCACACCGTGAGCAGTTG | SEQ ID No. 115 |
| CCGTCACACCGTGAGCAG | SEQ ID No. 116 |
| CTGATGCAAGCACCAGCT | SEQ ID No. 117 |
| CGGCGGACTCCGTAAAGG | SEQ ID No. 118 |
| GCTGATGCAAGCACCAGC | SEQ ID No. 119 |
| ACCGTCACACCGTGAGCA | SEQ ID No. 120 |
| CAGATGCAGACCAGACAG | SEQ ID No. 121 |
| TGATGCAAGCACCAGCTA | SEQ ID No. 122 |
| AGTTAGGAGACCTCGTTC | SEQ ID No. 123 |
| GGCGGACTCCGTAAAGGT | SEQ ID No. 124 |
| GTTAGGAGACCTCGTTCG | SEQ ID No. 125 |
| AGTTGCTCTCACGGTCGT | SEQ ID No. 126 |
| GCACCAGCTATCAGTTAG | SEQ ID No. 127 |
| TACCGTCACACCGTGAGC | SEQ ID No. 128 |
| AGATACCGTCACACCGTG | SEQ ID No. 129 |
| TAGATACCGTCACACCGT | SEQ ID No. 130 |
| TGCTCTCACGGTCGTTCT | SEQ ID No. 131 |
| ACCATGTGGTTCTCGTTG | SEQ ID No. 132 |
| ATGCAAGCACCAGCTATC | SEQ ID No. 133 |
| GGCGGCGGACTCCGTAAA | SEQ ID No. 134 |
| AGGCGGCGGACTCCGTAA | SEQ ID No. 135 |
| CACACCGTGAGCAGTTGC | SEQ ID No. 136 |
| TTAGATACCGTCACACCG | SEQ ID No. 137 |
| GAACCATGTGGTTCTCGT | SEQ ID No. 138 |
| GCTCTCACGGTCGTTCTT | SEQ ID No. 139 |
| CACCAGCTATCAGTTAGG | SEQ ID No. 140 |
| GCCACTCGGTCAGATCTA | SEQ ID No. 141 |
| GATACCGTCACACCGTGA | SEQ ID No. 142 |
| TCAGATGCAGACCAGACA | SEQ ID No. 143 |
| TAGGCGGCGGACTCCGTA | SEQ ID No. 144 |
| CCATGTGGTTCTCGTTGT | SEQ ID No. 145 |
| CAAGCACCAGCTATCAGT | SEQ ID No. 146 |

SEQ ID No. 108 to SEQ ID No. 146 are useful for the detection of L. lindneri.

| | |
|---|---|
| CGCTGAGTGGCGCGGGTT | SEQ ID No. 147 |
| CCGGATTCCGACGACGTT | SEQ ID No. 148 |
| CGCCAACCTTCCCAGATT | SEQ ID No. 149 |
| ACGACGTTTCACGTGTGT | SEQ ID No. 150 |
| CGACGACGTTTCACGTGT | SEQ ID No. 151 |
| CAAGTCCACAGTCTCGGT | SEQ ID No. 152 |
| CTACCCAGCGGTGGCGGT | SEQ ID No. 153 |
| AACCTGGCATGTTACCGT | SEQ ID No. 154 |
| GCGCACAGCACCCCTTCT | SEQ ID No. 155 |
| ACCAGTCCTTAACGGTCT | SEQ ID No. 156 |
| AGGTCAAGTCCACAGTCT | SEQ ID No. 157 |
| TTCCCCACGTCTACCTCT | SEQ ID No. 158 |
| TCCACTCCCAACCTATCT | SEQ ID No. 159 |
| GGGCTTCATTTCTGGGCT | SEQ ID No. 160 |
| GATTCTACGTCCGAGGCT | SEQ ID No. 161 |
| TGCACAACTTAGCCTCCT | SEQ ID No. 162 |
| CTTGCGCACAGCACCCCT | SEQ ID No. 163 |
| AGTTCCCCACGTCTACCT | SEQ ID No. 164 |
| GCTCCGGCTTTTAAACCT | SEQ ID No. 165 |
| AGCCTCCCCAGGAAACCT | SEQ ID No. 166 |
| GTTGGTTGCTTCCCTACT | SEQ ID No. 167 |
| GGCGGTGGCGGCGCAACT | SEQ ID No. 168 |
| CCCCACGTCTACCTCTAT | SEQ ID No. 169 |
| CTTCCACTCCCAACCTAT | SEQ ID No. 170 |
| TCGCCAACCTTCCCAGAT | SEQ ID No. 171 |
| TTGGTCCGCTCCGTACAT | SEQ ID No. 172 |
| GCTGTGTCAACACCCAAT | SEQ ID No. 173 |
| GCCAACCTTCCCAGATTG | SEQ ID No. 174 |
| GACGACGTTTCACGTGTG | SEQ ID No. 175 |
| TACCCAGCGGTGGCGGTG | SEQ ID No. 176 |
| GCACAACTTAGCCTCCTG | SEQ ID No. 177 |
| GCGGTGGCGGCGCAACTG | SEQ ID No. 178 |
| ACCCAGCGGTGGCGGTGG | SEQ ID No. 179 |
| CGGTGGCGGCGCAACTGG | SEQ ID No. 180 |
| TTGATTTCACCTACGGGG | SEQ ID No. 181 |
| CACGCTGAGTGGCGCGGG | SEQ ID No. 182 |
| AGGATCCTGAACTGAGGG | SEQ ID No. 183 |
| TCAAGTCCACAGTCTCGG | SEQ ID No. 184 |
| CAGCGGTGGCGGTGGCGG | SEQ ID No. 185 |
| CCACGCTGAGTGGCGCGG | SEQ ID No. 186 |
| TCCATACGGTACCACCGG | SEQ ID No. 187 |

SEQ ID Nos. 147 to 187 are useful for the detection of L. buchneri.

| | |
|---|---|
| CCGTCACGCCGACAACAG | SEQ ID No. 188 |
| ACCGTCACGCCGACAACA | SEQ ID No. 189 |
| ATACCGTCACGCCGACAA | SEQ ID No. 190 |
| TACCGTCACGCCGACAAC | SEQ ID No. 191 |
| GATACCGTCACGCCGACA | SEQ ID No. 192 |
| GGATACCGTCACGCCGAC | SEQ ID No. 193 |
| ACGCCGACAACAGTTACT | SEQ ID No. 194 |
| GGCTCGCTCCCTAAAAGG | SEQ ID No. 195 |
| CTCTGCCGACCATTCTTC | SEQ ID No. 196 |
| CTGCCGACCATTCTTCTC | SEQ ID No. 197 |
| CGCCGACAACAGTTACTC | SEQ ID No. 198 |
| CACGCCGACAACAGTTAC | SEQ ID No. 199 |
| TCACGCCGACAACAGTTA | SEQ ID No. 200 |
| TCTGCCGACCATTCTTCT | SEQ ID No. 201 |
| ACAACAGTTACTCTGCCG | SEQ ID No. 202 |
| CGGCTCGCTCCCTAAAAG | SEQ ID No. 203 |
| GACAACAGTTACTCTGCC | SEQ ID No. 204 |
| ACGGCTCGCTCCCTAAAA | SEQ ID No. 205 |
| CGACAACAGTTACTCTGC | SEQ ID No. 206 |
| CCGACAACAGTTACTCTG | SEQ ID No. 207 |
| ACTCTGCCGACCATTCTT | SEQ ID No. 208 |
| CTCGCTCCCTAAAAGGGT | SEQ ID No. 209 |
| TGCCGACCATTCTTCTCC | SEQ ID No. 210 |
| GCCGACCATTCTTCTCCA | SEQ ID No. 211 |
| CGCCATCTTTCAGCCAAG | SEQ ID No. 212 |
| GACGGCTCGCTCCCTAAA | SEQ ID No. 213 |
| CGACCATTCTTCTCCAAC | SEQ ID No. 214 |
| GTCACGCCGACAACAGTT | SEQ ID No. 215 |
| CCTGATCTCTCAGGTGAT | SEQ ID No. 216 |
| AACAGTTACTCTGCCGAC | SEQ ID No. 217 |
| TACTCTGCCGACCATTCT | SEQ ID No. 218 |
| CCGACCATTCTTCTCCAA | SEQ ID No. 219 |
| GCCGACAACAGTTACTCT | SEQ ID No. 220 |
| TTACTCTGCCGACCATTC | SEQ ID No. 221 |
| TCCCTAAAAGGGTTACGC | SEQ ID No. 222 |
| CAACAGTTACTCTGCCGA | SEQ ID No. 223 |
| AGACGGCTCGCTCCCTAA | SEQ ID No. 224 |
| ACGCCATCTTTCAGCCAA | SEQ ID No. 225 |
| AACCTGATCTCTCAGGTG | SEQ ID No. 226 |

SEQ ID Nos. 188 to 226 are useful for the detection of L. casei.

| | |
|---|---|
| ACTGCAAGCAGCTTCGGT | SEQ ID No. 227 |
| CGTCCACTGCAAGCAGCT | SEQ ID No. 228 |
| GTCAATCAACGTCCACTG | SEQ ID No. 229 |
| CACTGCAAGCAGCTTCGG | SEQ ID No. 230 |
| GTCTGAATGGTTATGCGG | SEQ ID No. 231 |
| TCGACGTCAGTGCGTTCG | SEQ ID No. 232 |
| CCACTGCAAGCAGCTTCG | SEQ ID No. 233 |
| TGCAAGCAGCTTCGGTCG | SEQ ID No. 234 |
| AAGCAGCTTCGGTCGACG | SEQ ID No. 235 |
| AACGTCCACTGCAAGCAG | SEQ ID No. 236 |
| GTCGACGTCAGTGCGTTC | SEQ ID No. 237 |
| TCCACTGCAAGCAGCTTC | SEQ ID No. 238 |
| GCAGCTTCGGTCGACGTC | SEQ ID No. 239 |
| TCAATCAACGTCCACTGC | SEQ ID No. 240 |
| ACGTCCACTGCAAGCAGC | SEQ ID No. 241 |
| TCAACGTCCACTGCAAGC | SEQ ID No. 242 |
| CAAGCAGCTTCGGTCGAC | SEQ ID No. 243 |
| CGACGTCAGTGCGTTCGA | SEQ ID No. 244 |
| GCAAGCAGCTTCGGTCGA | SEQ ID No. 245 |
| CAGCTTCGGTCGACGTCA | SEQ ID No. 246 |
| CAATCAACGTCCACTGCA | SEQ ID No. 247 |
| CAACGTCCACTGCAAGCA | SEQ ID No. 248 |
| GACGTCAGTGCGTTCGAC | SEQ ID No. 249 |
| GTCCACTGCAAGCAGCTT | SEQ ID No. 250 |
| ATCAACGTCCACTGCAAG | SEQ ID No. 251 |
| CCGTCAAAGGACTAACAG | SEQ ID No. 252 |
| GGTCTGAATGGTTATGCG | SEQ ID No. 253 |
| CGTCAATCAACGTCCACT | SEQ ID No. 254 |
| CAGTTACTCTAGTCCCTG | SEQ ID No. 255 |
| AGCTTCGGTCGACGTCAG | SEQ ID No. 256 |
| CTGCAAGCAGCTTCGGTC | SEQ ID No. 257 |
| CTAGTCCCTGTTCTTCTC | SEQ ID No. 258 |
| GGATACCGTCAAAGGACT | SEQ ID No. 259 |
| AGCAGCTTCGGTCGACGT | SEQ ID No. 260 |
| ACGTCAATCAACGTCCAC | SEQ ID No. 261 |
| GCTTCGGTCGACGTCAGT | SEQ ID No. 262 |
| ACGTCAGTGCGTTCGACT | SEQ ID No. 263 |
| ACCATGTGGTCTGAATGG | SEQ ID No. 264 |
| TCCCTAAAAGGGTTACCC | SEQ ID No. 265 |

SEQ ID Nos. 227 to 265 are useful for the detection of L. coryniformis.

| | |
|---|---|
| CTATCATTAGGCGCAGCT | SEQ ID No. 266 |
| ACTATCATTAGGCGCAGC | SEQ ID No. 267 |
| GGCGCAGCTCGTTCGACT | SEQ ID No. 268 |
| GCGGCAGGCTCCAAAAGG | SEQ ID No. 269 |
| ATTAGGCGCAGCTCGTTC | SEQ ID No. 270 |
| ATCATTAGGCGCAGCTCG | SEQ ID No. 271 |
| AGGCGCAGCTCGTTCGAC | SEQ ID No. 272 |
| CGGCAGGCTCCAAAAGGT | SEQ ID No. 273 |
| TCATTAGGCGCAGCTCGT | SEQ ID No. 274 |
| GCGCAGCTCGTTCGACTT | SEQ ID No. 275 |
| TTAGGCGCAGCTCGTTCG | SEQ ID No. 276 |
| GGCAGGCTCCAAAAGGTT | SEQ ID No. 277 |
| TATCATTAGGCGCAGCTC | SEQ ID No. 278 |
| GCAGGCTCCAAAAGGTTA | SEQ ID No. 279 |
| CGCAGCTCGTTCGACTTG | SEQ ID No. 280 |
| CATTAGGCGCAGCTCGTT | SEQ ID No. 281 |
| TAGGCGCAGCTCGTTCGA | SEQ ID No. 282 |
| TAGATACCGTCGCGACGT | SEQ ID No. 283 |
| TTAGATACCGTCGCGACG | SEQ ID No. 284 |
| ATACCGTCGCGACGTGAG | SEQ ID No. 285 |
| TACCGTCGCGACGTGAGC | SEQ ID No. 286 |
| GTTAGATACCGTCGCGAC | SEQ ID No. 287 |
| GATACCGTCGCGACGTGA | SEQ ID No. 288 |
| ACCGTCGCGACGTGAGCA | SEQ ID No. 289 |
| CAGGCTCCAAAAGGTTAC | SEQ ID No. 290 |
| CACGCCCGTTCTTCTCTA | SEQ ID No. 291 |
| GCGACGTGAGCAGTTACT | SEQ ID No. 292 |
| CGCGACGTGAGCAGTTAC | SEQ ID No. 293 |
| GCACAAAGGCCATCTTTC | SEQ ID No. 294 |
| AGGCGGCAGGCTCCAAAA | SEQ ID No. 295 |
| AGTTACTCTCACGCCCGT | SEQ ID No. 296 |
| GATAGCACAAAGGCCATC | SEQ ID No. 297 |
| TCGCGACGTGAGCAGTTA | SEQ ID No. 298 |
| CCACCTTAGGCGGCAGGC | SEQ ID No. 299 |
| ACCTTAGGCGGCAGGCTC | SEQ ID No. 300 |
| TAGGCGGCAGGCTCCAAA | SEQ ID No. 301 |
| GCAGTTACTCTCACGCCC | SEQ ID No. 302 |
| CGACGTGAGCAGTTACTC | SEQ ID No. 303 |
| CAGTTACTCTCACGCCCG | SEQ ID No. 304 |

The sequences SEQ ID Nos. 266 to 304 are useful for the detection of L. fructivorans.

| | |
|---|---|
| CCATGCGGTCTCCGTGGT | SEQ ID No. 305 |
| CATGCGGTCTCCGTGGTT | SEQ ID No. 306 |
| TGCGGTCTCCGTGGTTAT | SEQ ID No. 307 |
| GACCATGCGGTCTCCGTG | SEQ ID No. 308 |
| GGTGATGCAAGCACCACC | SEQ ID No. 309 |
| CATCTTTTACCCGGAGAC | SEQ ID No. 310 |
| ATGCGGTCTCCGTGGTTA | SEQ ID No. 311 |
| AGACCATGCGGTCTCCGT | SEQ ID No. 312 |
| GTGATGCAAGCACCACCG | SEQ ID No. 313 |
| ATTGGTGATGCAAGCACC | SEQ ID No. 314 |
| TGATGCAAGCACCACCGC | SEQ ID No. 315 |
| ACCATGCGGTCTCCGTGG | SEQ ID No. 316 |
| TTGGTGATGCAAGCACCA | SEQ ID No. 317 |
| CTTTTACCCGGAGACCAT | SEQ ID No. 318 |
| ATCTTTTACCCGGAGACC | SEQ ID No. 319 |
| TTACCCGGAGACCATGCG | SEQ ID No. 320 |
| TCTTTTACCCGGAGACCA | SEQ ID No. 321 |
| GGTCTCCGTGGTTATACG | SEQ ID No. 322 |
| GATGCAAGCACCACCGCA | SEQ ID No. 323 |
| GAGACCATGCGGTCTCCG | SEQ ID No. 324 |
| CGGTCTCCGTGGTTATAC | SEQ ID No. 325 |
| TTTACCCGGAGACCATGC | SEQ ID No. 326 |
| TGCAAGCACCACCGCAAA | SEQ ID No. 327 |
| GGAGACCATGCGGTCTCC | SEQ ID No. 328 |
| GCAAGCACCACCGCAAAC | SEQ ID No. 329 |
| TTTTACCCGGAGACCATG | SEQ ID No. 330 |
| AGCACCACCGCAAACTGA | SEQ ID No. 331 |
| AAGCACCACCGCAAACTG | SEQ ID No. 332 |
| CCATCTTTTACCCGGAGA | SEQ ID No. 333 |
| ATGCAAGCACCACCGCAA | SEQ ID No. 334 |
| GTCTCCGTGGTTATACGG | SEQ ID No. 335 |
| GCGGTCTCCGTGGTTATA | SEQ ID No. 336 |
| CAAGCACCACCGCAAACT | SEQ ID No. 337 |
| TCTCCGTGGTTATACGGT | SEQ ID No. 338 |
| CTCCGTGGTTATACGGTA | SEQ ID No. 339 |
| GCCATCTTTTACCCGGAG | SEQ ID No. 340 |
| CGCCATCTTTTACCCGGA | SEQ ID No. 341 |
| CAGCTGATCTCTCAGCCT | SEQ ID No. 342 |
| CGCAAACTGACCAAACCT | SEQ ID No. 343 |

SEQ ID Nos. 305 to 343 are useful for the detection of Lactobacillus perolens.

| | |
|---|---|
| AAGCTCGGACCATGCGGT | SEQ ID No. 344 |
| CTTTCAAGCTCGGACCAT | SEQ ID No. 345 |
| TTTCAAGCTCGGACCATG | SEQ ID No. 346 |
| GCCATCTTTCAAGCTCGG | SEQ ID No. 347 |
| CAAGCTCGGACCATGCGG | SEQ ID No. 348 |
| AGCCATCTTTCAAGCTCG | SEQ ID No. 349 |
| TCAAGCTCGGACCATGCG | SEQ ID No. 350 |
| AGCTCGGACCATGCGGTC | SEQ ID No. 351 |
| TTCAAGCTCGGACCATGC | SEQ ID No. 352 |
| CGAAGCCATCTTTCAAGC | SEQ ID No. 353 |
| GCTCGGACCATGCGGTCC | SEQ ID No. 354 |
| ATCTTTCAAGCTCGGACC | SEQ ID No. 355 |
| CATCTTTCAAGCTCGGAC | SEQ ID No. 356 |

SEQ ID Nos. 344 to 356 are useful for the detection of Lactobacillus plantarum.

| | |
|---|---|
| CAT GCG GCC TTT AGA TCG | SEQ ID No. 357 |
| TCC GAC ACT CCA GTC CGG | SEQ ID No. 358 |

SEQ ID Nos. 357 and 358 are especially useful for the detection of Megasphaera cerevisiae. SEQ ID No. 358 is a preferred embodiment of the invention.

| | |
|---|---|
| ATA GTG CCG TTC GTC CCC | SEQ ID No. 359 |
| TTG CTC CGG CAC AGA AAG | SEQ ID No. 360 |

SEQ ID Nos. 359 and 360 are especially useful for the detection of Pectinatus cerevisiiphilus.

| | |
|---|---|
| GCC CCT TAG CCG GCT TCG GG | SEQ ID No. 361 |
| GCGGCCCTTAGCCGGCTT | SEQ ID No. 362 |
| TGCGGCCCTTAGCCGGCT | SEQ ID No. 363 |
| CCTTGCGGCCCTTAGCCG | SEQ ID No. 364 |
| CGGCCCTTAGCCGGCTTC | SEQ ID No. 365 |
| TTGCGGCCCTTAGCCGGC | SEQ ID No. 366 |
| TGCGCCGTTACCGTCACC | SEQ ID No. 367 |
| GGCCCTTAGCCGGCTTCG | SEQ ID No. 368 |
| GCGCCGTTACCGTCACCA | SEQ ID No. 369 |
| CGCACTTTTAAGATCCGC | SEQ ID No. 370 |
| GTGCGCCGTTACCGTCAC | SEQ ID No. 371 |
| CGCCGTTACCGTCACCAA | SEQ ID No. 372 |
| AGACGGTCGGTGCCTTGC | SEQ ID No. 373 |
| GCCCTTAGCCGGCTTCGG | SEQ ID No. 374 |
| GGTGCGCCGTTACCGTCA | SEQ ID No. 375 |
| GACGGTCGGTGCCTTGCG | SEQ ID No. 376 |
| TGCCTTGCGGCCCTTAGC | SEQ ID No. 377 |
| GCCTTGCGGCCCTTAGCC | SEQ ID No. 378 |
| TGACCTGCGATTAGTAGC | SEQ ID No. 379 |
| CTTGCGGCCCTTAGCCGG | SEQ ID No. 380 |
| TGGTGCGCCGTTACCGTC | SEQ ID No. 381 |
| GACCTGCGATTAGTAGCG | SEQ ID No. 382 |
| CCTTAGCCGGCTTCGGGT | SEQ ID No. 383 |
| CCGCACTTTTAAGATCCG | SEQ ID No. 384 |
| CTGACCTGCGATTAGTAG | SEQ ID No. 385 |
| GTGCCTTGCGGCCCTTAG | SEQ ID No. 386 |
| ACGGTCGGTGCCTTGCGG | SEQ ID No. 387 |
| TACTGCCATTCGTCCCCT | SEQ ID No. 388 |
| GACCAGTTCGAATCCCAT | SEQ ID No. 389 |
| CCTCAGTTCGGACCCCAT | SEQ ID No. 390 |
| ACTGCCATTCGTCCCCTG | SEQ ID No. 391 |
| TTCGGACCCCATCACGGG | SEQ ID No. 392 |
| GTTCGGACCCCATCACGG | SEQ ID No. 393 |
| AGTTCGAATCCCATCACG | SEQ ID No. 394 |
| ACCAGTTCGAATCCCATC | SEQ ID No. 395 |
| CTCAGTTCGGACCCCATC | SEQ ID No. 396 |
| CTGCCATTCGTCCCCTGC | SEQ ID No. 397 |
| ATCCGCTTAATGTTCCGC | SEQ ID No. 398 |
| AAGCGACAGCTAAAAGCC | SEQ ID No. 399 |
| ATGACCAGTTCGAATCCC | SEQ ID No. 400 |

SEQ ID Nos. 361 to 400 are particularly useful for the detection of bacteria belonging to the genus Pectinatus.

| | |
|---|---|
| TCCAGGATCGGCTCCTTT | SEQ ID No. 401 |
| CTCCAGGATCGGCTCCTT | SEQ ID No. 402 |
| TCAGACGCAAACCCCTCT | SEQ ID No. 403 |
| GCTCCAGGATCGGCTCCT | SEQ ID No. 404 |
| CTCTTCCGGCGATAGACT | SEQ ID No. 405 |
| GCGGCCTTTAGATCGTAT | SEQ ID No. 406 |
| CTTCCGGCGATAGACTAT | SEQ ID No. 407 |
| CACGGCGTATGGGTATTG | SEQ ID No. 408 |
| GGTTTGCTCCAGGATCGG | SEQ ID No. 409 |
| CGCAAACCCCTCTTCCGG | SEQ ID No. 410 |
| GGGTTTGCTCCAGGATCG | SEQ ID No. 411 |
| TACGGTACCGTCACGGCG | SEQ ID No. 412 |
| ACGCAAACCCCTCTTCCG | SEQ ID No. 413 |
| CGGCGATAGACTATTCAG | SEQ ID No. 414 |
| GACACTCCAGTCCGGCAG | SEQ ID No. 415 |
| CCAGGATCGGCTCCTTTC | SEQ ID No. 416 |
| AGACGCAAACCCCTCTTC | SEQ ID No. 417 |
| TCCGGCGATAGACTATTC | SEQ ID No. 418 |
| ATCAGACGCAAACCCCTC | SEQ ID No. 419 |
| GTTTGCTCCAGGATCGGC | SEQ ID No. 420 |
| GCAAACCCCTCTTCCGGC | SEQ ID No. 421 |
| CCGACACTCCAGTCCGGC | SEQ ID No. 422 |
| CCTCTTCCGGCGATAGAC | SEQ ID No. 423 |
| TCTTCCGGCGATAGACTA | SEQ ID No. 424 |
| ACGGCGTATGGGTATTGA | SEQ ID No. 425 |
| CCGGCGATAGACTATTCA | SEQ ID No. 426 |
| CGACACTCCAGTCCGGCA | SEQ ID No. 427 |
| TCCGGCAGTTTCAATCCC | SEQ ID No. 428 |
| TGCTCCAGGATCGGCTCC | SEQ ID No. 429 |
| ATGCGGCCTTTAGATCGT | SEQ ID No. 430 |
| ATCCCTGGCACTCAATGT | SEQ ID No. 431 |
| AATCAGACGCAAACCCCT | SEQ ID No. 432 |
| CAAACCCCTCTTCCGGCG | SEQ ID No. 433 |
| TCATGCGGCCTTTAGATC | SEQ ID No. 434 |
| GACGCAAACCCCTCTTCC | SEQ ID No. 435 |
| TGCGGCCTTTAGATCGTA | SEQ ID No. 436 |
| TCTCTATCCCTGGCACTC | SEQ ID No. 437 |
| GGCTCCTTTCGCTTCCCT | SEQ ID No. 438 |
| CAGGATCGGCTCCTTTCG | SEQ ID No. 439 |

SEQ ID Nos. 401 to 439 are especially useful for the detection of Megasphaera cerevisiae.

| | |
|---|---|
| CCG CAC TTT TAA GAT CCG | SEQ ID No. 440 |

SEQ ID No. 440 is especially useful for the detection of the genus Pectinatus.

| | |
|---|---|
| GAT CCG CTT AGT CAT CCG | SEQ ID No. 441 |
| CTA CTG CCA TTC GTC CCC | SEQ ID No. 442 |

SEQ ID Nos. 441 and 442 are especially useful for the detection of Pectinatus cerevisiiphilus.

In the sequences K stands for “G+T”, M for “A+C”, R for “A+G”, “W for “A+T” and Y for “C+T”.

The subject of the present invention also comprises modifications of the aforementioned oligonucleotide sequences SEQ ID No. 1 to SEQ ID No. 442. These especially include:

a) nucleic acid molecules, which (i) are identical to one of the above oligonucleotide sequences (SEQ ID No. 1 to SEQ ID No. 442) in at least 80%, 84%, 87% and preferably in at least 90%, 92% and particularly preferably in at least 94, 96, 98% of the bases (wherein the sequence region of the nucleic acid molecule corresponding to the sequence region of one of the oligonucleotides given above (SEQ ID Nos. 1 to 442) is to be considered, and not the entire sequence of an oligonucleotide, which possibly may be longer in sequence compared to the oligonucleotides given above by one or numerous bases) or (ii) differing from the above oligonucleotide sequences by one or several deletions and/or additions, and which allow for a specific hybridization with nucleic acid sequences of the lactic-acid bacteria, which are harmful to beer, belonging to the genera of Lactobacillus and Pediococcus, especially to the species Pediococcus damnosus, Lactobacillus coryniformis, Lactobacillus perolens, Lactobacillus buchneri, Lactobacillus plantarum, Lactobacillus fructivorans, Lactobacillus lindneri, Lactobacillus casei, Lactobacillus brevis or of Gram-negative bacteria, which are harmful to beer, belonging to the genera of Pectinatus and Megasphaera, especially to the species of Pectinatus frisingensis, Pectinatus cerevisiiphilus, Megasphaera cerevisiae. “Specific hybridization” here means that under the hybridization conditions described here, or those known to the person skilled in the art in the context of in situ hybridization techniques, only the ribosomal RNA of target organisms binds to the oligonucleotide and not the rRNA of non-target organisms.

b) nucleic acid molecules, which hybridize under stringent conditions with a sequence being complementary to one of the oligonucleotides named under a) or to one of the probes identified in SEQ ID No. 1 to SEQ ID No. 442,

c) nucleic acid molecules comprising an oligonucleotide sequence from SEQ ID Nos. 1 to 442 or comprising the sequence of an oligonucleotide according to a) or b) and which, in addition to the sequences mentioned or their modifications according to a) or b), have at least one further nucleotide, and which allow for specific hybridization with nucleic acid sequences of target organisms.

The degree of sequence identity of a nucleic acid molecule with probes SEQ ID No. 1 to SEQ ID No. 442 can be determined by usual algorithms. In this respect, for example, the program for the determination of sequence identity which is accessible under hypertext transfer protocol on the worldwide web at “ncbi.nlm.nih.gov/BLAST” (http://www.ncbi.nlm.nih.gov/BLAST) (on this site there is for example the link “Standard nucleotide-nucleotide BLAST [blastn]”) is suitable here.

In the present invention “hybridization” can have the same meaning as “complementary”. The present invention also comprises those oligonucleotides, which hybridize to the (theoretical) antisense strand of one of the inventive oligonucleotides including the modifications of SEQ ID Nos. 1 to 442 according to the invention.

The nucleic acid probe molecules according to the invention can be used with various hybridization solutions in the context of the inventive detection method. For this purpose various organic solvents at concentrations of from 0 to 80% can be used. Compliance with stringent hybridization conditions ensures that the nucleic acid probe molecule will indeed hybridize with the target sequence. Moderate conditions within the meaning of the invention are, e.g. 0% formamide in a hybridization buffer as described below. Stringent conditions within the meaning of the invention are for example 20-80% formamide in the hybridization buffer.

Moreover, stringent hybridization conditions may of course also be looked up in the literature and standard works of reference (such as the Manual of Sambrook et al. (1989) Cold Spring Harbor Laboratory Press, Cold Spring Harbor, N.Y.). Generally, “specifically hybridizing” means that a molecule preferentially binds to a specific nucleic sequence under stringent conditions when this sequence is present in a complex mixture of (for example total) DNA or RNA. The term “stringent conditions” stands for conditions under which a probe preferentially hybridizes to its target sequence, and to a clearly smaller extent, or not at all, to other sequences. Stringent conditions are partly sequence-dependent and will vary under different circumstances. Longer sequences hybridize specifically at higher temperatures. In general, stringent conditions are selected in such a way that the temperature is approximately 5° C. below the thermal melting point (Tₘ) for the specific sequence at a defined ionic strength and a defined pH. Tₘ stands for the temperature (under defined ionic strength, pH and nucleic acid concentration), at which 50% of the probe molecules complementary to the target sequence hybridize to the target sequence in the equilibrium state. (Due to the fact that target sequences are usually in excess, in equilibrium 50% of the probes are involved). Typically, stringent conditions are those at which the salt concentration is at least about 0.01 to 1.0 M of sodium ions (or another salt) at a pH between 7.0 and 8.3 and the temperature is at least about 30° C. for short probes (i.e. for example 10-50 nucleotides). Additionally, stringent conditions as already mentioned above may be achieved by the addition of destabilizing agents such as formamide.

Within the scope of the method according to the invention, a typical hybridization solution contains 0-80% formamide, preferably 20-60% formamide and especially preferably 35% formamide and has a salt concentration of 0.1 mol/l to 1.5 mol/l, preferably 0.5 mol/l to 1.0 mol/l, more preferably from 0.7 mol/1-0.9 mol/l, most preferably of 0.9 mol/l, with the salt being preferably sodium chloride. Further, the hybridization solution usually comprises a detergent, such as for instance sodium lauryl sulfate (SDS) in a concentration of 0.001% to 0.2%, preferably in a concentration of 0.005-0.05%, more preferably 0.01-0.03%, and most preferably of 0.01%. The hybridization solution may be buffered with various compounds, such as tris-HCl, sodium citrate, PIPES or HEPES buffer, which are used usually at concentrations of from 0.01-0.1 mol/l, preferably from 0.01 to 0.08 mol/l, in a pH range of from 6.0 to 9.0, preferably 7.0 to 8.0. The preferred embodiment of the hybridization solution of the present invention contains 0.02 mol/l tris-HCl, pH 8.0.

It shall be understood that the person skilled in the art can select the given concentrations of the components of the hybridization buffer in such a way that the required stringency of the hybridization reaction is achieved. Particularly preferred embodiments reflect stringent to particularly stringent hybridization conditions. Using these stringent conditions, the person skilled in the art can determine whether a given nucleic acid molecule permits the specific detection of nucleic acid sequences of target organisms and can therefore be used reliably in the context of the invention.

The concentration of the probe may vary greatly depending on the type of labeling and the number of the target structures expected. To allow rapid and efficient hybridization, the number of nucleic acid probe molecules should exceed the number of target structures by several orders of magnitude. On the other hand, it needs to be considered when working with fluorescence in situ hybridization (FISH) that an excessively high level of a fluorescently labeled hybridization probe leads to an increase in background fluorescence. The concentration of the probe should therefore be in the range of 0.5 ng/μl and 500 ng/μl, preferably between 1.0 ng/μl and 100 ng/μl and particularly preferably in the range of 1.0-50 ng/μl.

In the context of the method of the present invention, the preferred concentration is 1-10 ng of each nucleic acid molecule used per μl hybridization solution. The used volume of the hybridization solution should be between 8 μl and 100 ml; in a particularly preferred embodiment of the method of the present invention it is 40 μl.

The duration of the hybridization is normally between 10 minutes and 12 hours; the hybridization preferably lasts for about 1.5 hours. The hybridization temperature is preferably between 44° C. and 48° C., particularly preferably 46° C., whereby the parameter of the hybridization temperature as well as the concentration of salts and detergents in the hybridization solution can be optimized based on the nucleic acid probes, in particular their lengths and the degree to which they are complementary to the target sequence within the cell to be detected. The person skilled in the art is familiar with the pertinent calculations.

After completion of hybridization, the non-hybridized and excess nucleic acid probe molecules should be removed or washed off, which step is usually accomplished by a conventional washing solution. If desired, this washing solution can contain 0.001-0.1% of a detergent such as SDS, a concentration of 0.01% being preferred, as well as tris-HCl in a concentration of 0.001-0.1 mol/l, preferably 0.01-0.05 mol/l, more preferably 0.02 mol/l, the pH value of tris-HCl being in the range of 6.0 to 9.0, preferably at 7.0 to 8.0, and more preferably at 8.0. A detergent can be included, but it is not mandatory. The washing solution also usually contains NaCl at a concentration depending on the required stringency, of 0.003 mol/l to 0.9 mol/l, preferably from 0.01 mol/l to 0.9 mol/l. An NaCl concentration is particularly preferably about 0.07 mol/l. In addition, the washing solution may contain EDTA at a concentration up to 0.01 mol/l, the concentration preferably being 0.005 mol/l. The washing solution can further contain suitable quantities of commonly used preservatives which are known to the person skilled in the art.

In general, buffer solutions are used in the washing step, which can in principle be very similar to the hybridization buffer (buffered sodium chloride solution), the only difference being that the washing step is usually performed in a buffer with lower salt concentrations or at higher temperature.

The following equation can be used for the theoretical estimation of the hybridization conditions:

Td=81.5+16.6 lg[Na+]+0.4×(% GC)−820/n−0.5×(% FA)

Td=dissociation temperature in ° C.

[Na+]=molarity of sodium ions

% GC=proportion of guanine and cytosine nucleotides relative to the number of total bases

n=hybrid length

% FA=formamide content

Using this equation, for example the proportion of formamide in the washing buffer (which should be kept as low as possible because of formamide's toxicity) can be replaced with a correspondingly lower content of sodium chloride. However, the person skilled in the art is aware, on the basis of the extensive literature on in situ hybridization and methods, that these components can be varied as well as how they can be varied. The above remarks with respect to hybridization buffers also apply to the stringency of the hybridization conditions.

The “washing off” of the unbound nucleic acid probe molecules is normally accomplished at temperatures in the range of from 44° C. to 52° C., preferably from 44° C. to 50° C. and particularly preferably at 46° C. for a duration of 10-40 minutes, preferably for 15 minutes.

In an alternative embodiment of the method of the present invention, the nucleic acid probe molecules according to the invention are used in the so-called Fast FISH method for specifically detecting the given target organisms. The Fast FISH method is known to the person skilled in the art and is, for example, described in German patent application DE 199 36 875.9 and in international application WO 99/18234. Explicit reference is made here to the disclosure for performing the detection method described in these documents.

The specifically hybridized nucleic acid probe molecules can then be detected in the corresponding cells, provided that the nucleic acid probe molecule is detectable, for instance in that the nucleic acid probe molecule is covalently linked to a marker. Detectable markers which are used and which are all well known to the person skilled in the art include fluorescent groups such as CY2 (available from Amersham Life Sciences, Inc., Arlington Heights, USA), CY3 (also available from Amersham Life Sciences), CY5 (also available from Amersham Life Sciences), FITC (Molecular Probes Inc., Eugene, USA), FLUOS (available from Roche Diagnostics GmbH, Mannheim, Germany), TRITC (available from Molecular Probes Inc., Eugene, USA) or FLUOS-PRIME. Chemical markers, radioactive markers or enzymatic markers such as horseradish peroxidase, acid phosphatase, alkaline phosphatase and peroxidase can be used as well. A series of chromogens is known for each of these enzymes, which can be reacted instead of the natural substrate, forming colored or fluorescent products. Examples of such chromogens are given in the following Table:

TABLE 1

| Enzyme | Chromogen |
|---|---|
| 1. Alkaline phosphatase | 4-methylumbelliferylphosphate (*), |
| and acid phosphatase | bis(4-methylumbelliferylphosphate), (*) 3-O- |
| | methylfluorescein, flavone-3- |
| | diphosphate triammonium salt (*), |
| | p-nitrophenylphosphate disodium salt |
| 2. Peroxidase | tyramine hydrochloride (*), 3-(p- |
| | hydroxyphenyl)-propionic acid(*), |
| | p-hydroxyphenethylalcohol(*), |
| | 2,2′-azino-di-3-ethylbenzthiazolinesulfonic acid |
| | (ABTS), ortho-phenylendiamine dihydro- |
| | chloride, o-dianisidine, 5-aminosalicylic acid, |
| | p-ucresol(*), 3,3′-dimethyloxybenzidine, |
| | 3-methyl-2-benzothiazoline hydrazone, |
| | tetramethylbenzidine |
| 3. Horseradish | H₂O₂ + diammonium benzidine |
| peroxidase | H₂O₂ + tetramethylbenzidine |
| 4. β-D-galactosidase | o-Nitrophenyl-β-D-galactopyranoside, |
| | 4-methylumbelliferyl-β-D-galactoside |
| 5. Glucose oxidase | ABTS, glucose and thiazolyl blue |

*Fluorescence

Finally, it is possible to form nucleic acid probe molecules in such a way that there is a further nucleic acid sequence at their 5′ or 3′ end, which is also suitable for hybridization. This nucleic acid sequence in turn includes approximately 15 to 1,000, preferably 15-50 nucleotides. This second nucleic acid region can then be recognized by a nucleic acid probe molecule, which is detectable by any of the agents given above.

Another possibility is the coupling of the detectable nucleic acid probe molecules to a hapten. This nucleic acid probe molecule can then be brought into contact with antibodies, which recognize the hapten. An example of such a hapten is digoxigenin. Further examples besides those mentioned above are well known to the person skilled in the art.

The final analysis depends on the type of labeling of the used probe and can be conducted using an optical microscope, an epifluoresence microscope, chemoluminometer, fluorometer or the like.

An important advantage of the method described in this application for the specific and fast detection of bacteria which are harmful to beer is its speed compared to conventional detection methods as described above. In comparison to conventional cultivation methods, which require seven to twelve days for the detection, results using the method of the present invention are available within 48 hours.

Another advantage is the simultaneous detection of all relevant lactic-acid bacteria, which are harmful to beer as well as the opportunity of detecting Gram-negative beer contaminants at the same time.

Another advantage is the ability to differentiate between various species of the genus Lactobacillus. This is rendered possible easily and reliably using various labeled nucleic acid probe molecules.

Another advantage is the specificity of the method. With the used nucleic acid probe molecules, specifically the genus Lactobacillus and highly specifically the species Lactobacillus coryniformis, Lactobacillus perolens, Lactobacillus buchneri, Lactobacillus plantarum, Lactobacillus fructivorans, Lactobacillus lindneri, Lactobacillus casei, Lactobacillus brevis as well as in addition the species Pediococcus damnosus can be detected and visualized. It is likewise possible to detect and visualize specifically the genus Pectinatus and highly specifically the species Pectinatus frisingensis and Pectinatus cerevisiiphilus as well as the species Megasphaera cerevisiae. By visualization of the bacteria, a visual control may be carried out at the same time. False positive results are thus excluded.

Altogether the opportunity of simultaneous detection of the named germs represents an important advantage over the state of the art. The use of appropriate mixtures of probes, especially of those probes mentioned above as preferred probes, renders possible for example simultaneous detection of all named germs. This is an enormous advantage, because all bacteria which are relevant in practice and which are harmful to beer are evaluated in a single step.

A further advantage over the prior art is real saving in time. Hybridization in the state of the art normally takes 4 hours, whereas the method according to the invention only takes 1.5 hours.

A further advantage of the inventive method is its ease of handling, so that large amounts of samples may be tested for the presence of the mentioned bacteria.

The method of the present invention can be applied in a variety of ways.

Clear beers as well as beers, which are turbid due to the presence of yeast, may be analyzed, and in addition for example yeast samples (pure culture yeasts; “primary grown” yeasts i.e. harvest yeasts; or brewer's yeasts and yeast sediments) and rinsing water.

Another field of application for the inventive method is the microbiological analysis of all foodstuffs in which the detected bacteria play a role as food contaminants, which cause food spoilage.

Furthermore, according to the invention, a kit for performing the method according to the invention is provided. The hybridization arrangement contained in these kits is, for instance, described in German patent application 100 61 655.0. It is expressly referred to the disclosure contained in this document concerning the in situ hybridization arrangement.

Besides the described hybridization arrangement (called VIT reactor), the most important component of the kits is their respective hybridization solution containing the specific nucleic acid probe molecules for the microorganisms to be detected, as described above (so-called VIT solution). The kits also always contain the corresponding hybridization buffer (Solution C) and a concentrate of the corresponding washing solution (Solution D). The kit may also contain fixation solutions (Solution A and Solution B) if needed, and additionally a cell breaking solution (Breaker_{—}2) as well as, if needed, an embedding solution (finisher). Finishers are commercially available and their activity also includes the prevention of rapid bleaching of fluorescent probes under the fluorescent microscope. Optionally, solutions for parallel performance of a positive control and a negative control may also be included.

The following example is intended to describe the invention, however without limiting it:

### EXAMPLE

Specific fast detection of the bacteria, which are harmful to beer, in a sample

A sample is properly cultivated for 20-44 hours (e.g. in NBB medium, 48 hours, 28° C.).

An aliquot of the culture is centrifuged (5 min, 8000 rpm, room temperature), the supernatant is discarded, and the pellet is dissolved in a suitable volume of fixation solution (Solution A, 50% ethanol).

Thereafter an appropriate aliquot of the fixed cells (40 μl are preferred) is applied onto a slide and dried (46° C., 30 min or until completely dried).

The dried cells are then completely dehydrated by adding a further fixation solution (Solution B, absolute ethanol, 40 μl are preferred). The slide is again dried (room temperature, 3 min or until totally dry). For complete disintegration of the cells, a suitable volume of the cell breaking solution (Breaker_{—}2, 40 μl are preferred) is applied onto the slide and the slide is incubated (10 min, room temperature).

The cell disintegrating or breaking solution is washed off by immersing the slide in a tube, preferably the VIT reactor, filled with distilled water and the slide is then dried in a lateral position.

Thereinafter the hybridization solution (VIT solution) comprising the specific nucleic acid probe molecules described above for each of the microorganisms to be detected is applied onto the fixed dehydrated cells. The preferred volume is 40 μl.

The slide is then incubated within a chamber, preferably the VIT reactor (46° C., 90 min) which is moistened with hybridization buffer (Solution C, which corresponds to the hybridization solution without oligonucleotides).

The slide is then removed from the chamber, the chamber is filled with washing solution (Solution D, diluted 1:10 in distilled water) and the slide is incubated therein (46° C., 15 min).

The chamber is then filled with distilled water, the slide is immersed in it for a short period and is subsequently air-dried in a lateral position (46° C., 30 min or until completely dry).

The slide is then embedded in a suitable medium (finisher).

Finally, the sample is analyzed using a fluorescence microscope.

What is claimed is:

1. An isolated oligonucleotide having the sequence of any one of SEQ ID NOs. 1-442.

2. A method for detecting bacteria in a sample, comprising the steps:
cultivating the bacteria contained in the sample;
fixing the bacteria contained in the sample;
incubating the fixed cells with at least one oligonucleotide having a sequence of any of SEQ ID NOs. 1-442, in order to achieve hybridization;
removing or washing off the non-hybridized oligonucleotides; and
detecting the hybridized oligonucleotide, thereby detecting the bacteria.

3. The method according to claim 2, wherein the sample is a beer sample, a yeast sample or a rinse water sample.

4. The method according to claim 2, wherein the sample is a food sample.

5. The method according to claim 2, wherein the bacteria is a lactic-acid bacteria or a Gram-negative bacteria.

6. The method according to claim 5, wherein the lactic-acid bacteria or the Gram-negative bacteria is selected from the group consisting of Lactobacillus, Pediococcus, Pectinatus and Megasphaera.

7. The method according to claim 6, wherein the Lactobacillus, Pediococcus, Pectinatus or Megasphaera is selected from the group consisting of Pediococcus damnosus, Lactobacillus coryniformis, Lactobacillus perolens, Lactobacillus buchneri, Lactobacillus plantarum, Lactobacillus fructivorans, Lactobacillus lindneri, Lactobacillus casei, Lactobacillus brevis, Pectinatus frisingensis, Pectinatus cerevisiiphilus and Megasphaera cerevisiae.

8. The method according to claim 5, wherein the sample is a beer sample, a yeast sample or a rinse water sample.

9. The method according to claim 5, wherein the sample is a food sample.

10. The method according to claim 2, further comprising quantifying and visualizing the bacteria with hybridized oligonucleotides.

11. A method for the detection of a bacteria which is harmful to beer in a sample using an oligonucleotide according to claim 1.

12. The method according to claim 11, wherein the bacteria which is harmful to beer is a lactic-acid bacteria or a Gram-negative bacteria.

13. The method according to claim 12, wherein the lactic-acid bacteria or the Gram-negative bacteria is selected from the group consisting of Lactobacillus, Pediococcus, Pectinatus and Megasphaera.

14. The method according to claim 13, wherein the Lactobacillus, Pediococcus, Pectinatus or Megasphaera is selected from the group consisting of Pediococcus damnosus, Lactobacillus coryniformis, Lactobacillus perolens, Lactobacillus buchneri, Lactobacillus plantarum, Lactobacillus fructivorans, Lactobacillus lindneri, Lactobacillus casei, Lactobacillus brevis, Pectinatus frisingensis, Pectinatus cerevisiiphilus and Megasphaera cerevisiae.

15. A kit for performing the method according claim 2, containing at least one oligonucleotide according to claim 1.

16. The kit according to claim 15, which contains at least one oligonucleotide in a hybridization solution.

17. The kit according to claim 15, further containing a washing solution.

18. The kit according to claim 15, further comprising one or more fixation solutions.

19. The kit according to claim 15, further comprising a cell breaking solution or enzyme solution.

20. A kit for performing the method according to claim 5, containing at least one oligonucleotide according to claim 1.

21. The kit according to claim 20, which contains at least one oligonucleotide in a hybridization solution.

22. The kit according to claim 20, further comprising a washing solution.

23. The kit according to claim 20, further comprising one or more fixation solutions.

24. The kit according to claim 20, further comprising a cell breaking solution or enzyme solution.


SEQUENCE LISTING (442 sequences)

SEQ ID NO: 1 - DNA, 18 bp, Artificial Sequence
  Probe for the detection of Lactobacillus perolens
tggtgatgcaagcaccac

SEQ ID NO: 2 - DNA, 19 bp, Artificial Sequence
  Probe for the detection of Lactobacillus perolens
atgmtgatgcaagcaccar

SEQ ID NO: 3 - DNA, 18 bp, Artificial Sequence
  Probe for the detection of Lactobacillus perolens
catgcggtctccgtggtt

SEQ ID NO: 4 - DNA, 18 bp, Artificial Sequence
  Probe for the detection of Lactobacillus buchneri
acgctgagtggcgcgggt

SEQ ID NO: 5 - DNA, 19 bp, Artificial Sequence
  Probe for the detection of Lactobacillus plantarum
gcgggaccatccaaaagtg

SEQ ID NO: 6 - DNA, 18 bp, Artificial Sequence
  Probe for the detection of Lactobacillus fructivorans
ggcggcagggtccaaaag

SEQ ID NO: 7 - DNA, 18 bp, Artificial Sequence
  Probe for the detection of Lactobacillus casei
cgtcacgccgacaacagt

SEQ ID NO: 8 - DNA, 18 bp, Artificial Sequence
  Probe for the detection of Lactobacillus coryniformis
ggcggctagttccctaaa

SEQ ID NO: 9 - DNA, 20 bp, Artificial Sequence
  Probe for the detection of Lactobacillus brevis
accgtcaacccttgaacagt

SEQ ID NO: 10 - DNA, 18 bp, Artificial Sequence
  Probe for the detection of Lactobacillus brevis
gactcccgaaggttatct

SEQ ID NO: 11 - DNA, 18 bp, Artificial Sequence
  Probe for the detection of Lactobacillus lindneri
tcggtcagatctatcgtc

SEQ ID NO: 12 - DNA, 18 bp, Artificial Sequence
  Probe for the detection of Pediococcus damnosus
gctacgtatcacagcctt

SEQ ID NO: 13 - DNA, 18 bp, Artificial Sequence
  Probe for the detection of Lactobacillus lindner
gcggcggactccgtaaag

SEQ ID NO: 14 - DNA, 18 bp, Artificial Sequence
  Probe for the detection of the genera Pediococcus and Lactobacillus
gctacccaygctttcgag

SEQ ID NO: 15 - DNA, 18 bp, Artificial Sequence
  Probe for the detection of bacteria belonging to the genus of Pediococcus, especially P. acidilactici, P. pentosaceus, P. damnosus and P. parvulus
ccaatgcacttcttcggt

SEQ ID NO: 16 - DNA, 18 bp, Artificial Sequence
  Probe for the detection of L. casei
gctcgctccctaaaaggc

SEQ ID NO: 17 - DNA, 18 bp, Artificial Sequence
  Probe for the detection of L. coryniformis
actgcaagcagcttcggt

SEQ ID NO: 18 - DNA, 18 bp, Artificial Sequence
  Probe for the detection of L. fructivorans
cgccgcggatccatccaa

SEQ ID NO: 19 - DNA, 18 bp, Artificial Sequence
  Probe for the detection of P. damnosus
tgctttcgagacctcagc

SEQ ID NO: 20 - DNA, 18 bp, Artificial Sequence
  Probe for the detection of P. damnosus
ttacaagaccagacagcc

SEQ ID NO: 21 - DNA, 20 bp, Artificial Sequence
  Probe for the detection of L. brevis
accgtcaacccttgaacagt

SEQ ID NO: 22 - DNA, 18 bp, Artificial Sequence
  Probe for the detection of L. plantarum
acgccgcgggaccatcca

SEQ ID NO: 23 - DNA, 18 bp, Artificial Sequence
  Probe for the detection of L. plantarum
agttcgccactcactcaa

SEQ ID NO: 24 - DNA, 19 bp, Artificial Sequence
  Probe for the detection of the genera of Pediococcus and Lactobacillus
cgctacccatgctttcgkg

SEQ ID NO: 25 - DNA, 20 bp, Artificial Sequence
  Probe for the detection of the genera of Pediococcus and Lactobacillus
ccactacccatgctttcgag

SEQ ID NO: 26 - DNA, 18 bp, Artificial Sequence
  Probe for the detection of L. lindneri
caagcaccagctatcagt

SEQ ID NO: 27 - DNA, 18 bp, Artificial Sequence
  Probe for the detection of L. brevis
acgtcattcaacggaagc

SEQ ID NO: 28 - DNA, 18 bp, Artificial Sequence
  Probe for the detection of P. damnosus
agcttcgatgcaagcatc

SEQ ID NO: 29 - DNA, 18 bp, Artificial Sequence
  Probe for the detection of P. damnosus
tacaagaccagacagccg

SEQ ID NO: 30 - DNA, 18 bp, Artificial Sequence
  Probe for the detection of P. damnosus
gttacaagaccagacagc

SEQ ID NO: 31 - DNA, 18 bp, Artificial Sequence
  Probe for the detection of P. damnosus
acaagaccagacagccgc

SEQ ID NO: 32 - DNA, 18 bp, Artificial Sequence
  Probe for the detection of P. damnosus
cgtcagttacaagaccag

SEQ ID NO: 33 - DNA, 18 bp, Artificial Sequence
  Probe for the detection of P. damnosus
gcgtcagttacaagacca

SEQ ID NO: 34 - DNA, 18 bp, Artificial Sequence
  Probe for the detection of P. damnosus
gagacctcagcgtcagtt

SEQ ID NO: 35 - DNA, 18 bp, Artificial Sequence
  Probe for the detection of P. damnosus
agcgtcagttacaagacc

SEQ ID NO: 36 - DNA, 18 bp, Artificial Sequence
  Probe for the detection of P. damnosus
caagaccagacagccgcc

SEQ ID NO: 37 - DNA, 18 bp, Artificial Sequence
  Probe for the detection of P. damnosus
acccatgctttcgagacc

SEQ ID NO: 38 - DNA, 18 bp, Artificial Sequence
  Probe for the detection of P. damnosus
acgtattaccgcggctcg

SEQ ID NO: 39 - DNA, 18 bp, Artificial Sequence
  Probe for the detection of P. damnosus
taaaaaaaccgcctgcgc

SEQ ID NO: 40 - DNA, 18 bp, Artificial Sequence
  Probe for the detection of P. damnosus
atgctttcgagacctcag

SEQ ID NO: 41 - DNA, 18 bp, Artificial Sequence
  Probe for the detection of P. damnosus
ccatgctttcgagacctc

SEQ ID NO: 42 - DNA, 18 bp, Artificial Sequence
  Probe for the detection of P. damnosus
tgctttcgagacctcagc

SEQ ID NO: 43 - DNA, 18 bp, Artificial Sequence
  Probe for the detection of P. damnosus
catgctttcgagacctca

SEQ ID NO: 44 - DNA, 18 bp, Artificial Sequence
  Probe for the detection of P. damnosus
cccatgctttcgagacct

SEQ ID NO: 45 - DNA, 18 bp, Artificial Sequence
  Probe for the detection of P. damnosus
agacctcagcgtcagtta

SEQ ID NO: 46 - DNA, 18 bp, Artificial Sequence
  Probe for the detection of P. damnosus
ctttcgagacctcagcgt

SEQ ID NO: 47 - DNA, 18 bp, Artificial Sequence
  Probe for the detection of P. damnosus
cgagacctcagcgtcagt

SEQ ID NO: 48 - DNA, 18 bp, Artificial Sequence
  Probe for the detection of P. damnosus
gctttcgagacctcagcg

SEQ ID NO: 49 - DNA, 18 bp, Artificial Sequence
  Probe for the detection of P. damnosus
tcgagacctcagcgtcag

SEQ ID NO: 50 - DNA, 18 bp, Artificial Sequence
  Probe for the detection of P. damnosus
tttcgagacctcagcgtc

SEQ ID NO: 51 - DNA, 18 bp, Artificial Sequence
  Probe for the detection of P. damnosus
ttcgagacctcagcgtca

SEQ ID NO: 52 - DNA, 18 bp, Artificial Sequence
  Probe for the detection of P. damnosus
tacgtattaccgcggctc

SEQ ID NO: 53 - DNA, 18 bp, Artificial Sequence
  Probe for the detection of P. damnosus
aaaaaaaccgcctgcgct

SEQ ID NO: 54 - DNA, 18 bp, Artificial Sequence
  Probe for the detection of P. damnosus
gcttcgatgcaagcatct

SEQ ID NO: 55 - DNA, 18 bp, Artificial Sequence
  Probe for the detection of P. damnosus
cagcttcgatgcaagcat

SEQ ID NO: 56 - DNA, 18 bp, Artificial Sequence
  Probe for the detection of P. damnosus
atcagcttcgatgcaagc

SEQ ID NO: 57 - DNA, 18 bp, Artificial Sequence
  Probe for the detection of P. damnosus
tcagcttcgatgcaagca

SEQ ID NO: 58 - DNA, 18 bp, Artificial Sequence
  Probe for the detection of P. damnosus
cagcgtcagttacaagac

SEQ ID NO: 59 - DNA, 18 bp, Artificial Sequence
  Probe for the detection of P. damnosus
aagaccagacagccgcct

SEQ ID NO: 60 - DNA, 18 bp, Artificial Sequence
  Probe for the detection of P. damnosus
tacccatgctttcgagac

SEQ ID NO: 61 - DNA, 18 bp, Artificial Sequence
  Probe for the detection of P. damnosus
tagctcccgaaggttact

SEQ ID NO: 62 - DNA, 18 bp, Artificial Sequence
  Probe for the detection of P. damnosus
cgaaggttactccaccgg

SEQ ID NO: 63 - DNA, 18 bp, Artificial Sequence
  Probe for the detection of P. damnosus
ccgaaggttactccaccg

SEQ ID NO: 64 - DNA, 18 bp, Artificial Sequence
  Probe for the detection of P. damnosus
gctcccgaaggttactcc

SEQ ID NO: 65 - DNA, 18 bp, Artificial Sequence
  Probe for the detection of P. damnosus
cccgaaggttactccacc

SEQ ID NO: 66 - DNA, 18 bp, Artificial Sequence
  Probe for the detection of P. damnosus
tcccgaaggttactccac

SEQ ID NO: 67 - DNA, 18 bp, Artificial Sequence
  Probe for the detection of P. damnosus
ctcccgaaggttactcca

SEQ ID NO: 68 - DNA, 18 bp, Artificial Sequence
  Probe for the detection of L. brevis
ccgtcaacccttgaacag

SEQ ID NO: 69 - DNA, 18 bp, Artificial Sequence
  Probe for the detection of L. brevis
cattcaacggaagctcgt

SEQ ID NO: 70 - DNA, 18 bp, Artificial Sequence
  Probe for the detection of L. brevis
accgtcaacccttgaaca

SEQ ID NO: 71 - DNA, 18 bp, Artificial Sequence
  Probe for the detection of L. brevis
cttagcctcacgacttcg

SEQ ID NO: 72 - DNA, 18 bp, Artificial Sequence
  Probe for the detection of L. brevis
taccgtcaacccttgaac

SEQ ID NO: 73 - DNA, 18 bp, Artificial Sequence
  Probe for the detection of L. brevis
aacggaagctcgttcgac

SEQ ID NO: 74 - DNA, 18 bp, Artificial Sequence
  Probe for the detection of L. brevis
ttagcctcacgacttcgc

SEQ ID NO: 75 - DNA, 18 bp, Artificial Sequence
  Probe for the detection of L. brevis
gcaagcacgtcattcaac

SEQ ID NO: 76 - DNA, 18 bp, Artificial Sequence
  Probe for the detection of L. brevis
tcgccactcgcttcattg

SEQ ID NO: 77 - DNA, 18 bp, Artificial Sequence
  Probe for the detection of L. brevis
tcaacggaagctcgttcg

SEQ ID NO: 78 - DNA, 18 bp, Artificial Sequence
  Probe for the detection of L. brevis
ttcaacggaagctcgttc

SEQ ID NO: 79 - DNA, 18 bp, Artificial Sequence
  Probe for the detection of L. brevis
caagcacgtcattcaacg

SEQ ID NO: 80 - DNA, 18 bp, Artificial Sequence
  Probe for the detection of L. brevis
cacgtcattcaacggaag

SEQ ID NO: 81 - DNA, 18 bp, Artificial Sequence
  Probe for the detection of L. brevis
tcattcaacggaagctcg

SEQ ID NO: 82 - DNA, 18 bp, Artificial Sequence
  Probe for the detection of L. brevis
tgactcccgaaggttatc

SEQ ID NO: 83 - DNA, 18 bp, Artificial Sequence
  Probe for the detection of L. brevis
cgtcattcaacggaagct

SEQ ID NO: 84 - DNA, 18 bp, Artificial Sequence
  Probe for the detection of L. brevis
gcttagcctcacgacttc

SEQ ID NO: 85 - DNA, 18 bp, Artificial Sequence
  Probe for the detection of L. brevis
ttcgccactcgcttcatt

SEQ ID NO: 86 - DNA, 18 bp, Artificial Sequence
  Probe for the detection of L. brevis
gtcattcaacggaagctc

SEQ ID NO: 87 - DNA, 18 bp, Artificial Sequence
  Probe for the detection of L. brevis
cctgcttctgggcagatt

SEQ ID NO: 88 - DNA, 18 bp, Artificial Sequence
  Probe for the detection of L. brevis
ctgcttctgggcagattt

SEQ ID NO: 89 - DNA, 18 bp, Artificial Sequence
  Probe for the detection of L. brevis
gcacgtcattcaacggaa

SEQ ID NO: 90 - DNA, 18 bp, Artificial Sequence
  Probe for the detection of L. brevis
caacggaagctcgttcga

SEQ ID NO: 91 - DNA, 18 bp, Artificial Sequence
  Probe for the detection of L. brevis
acggaagctcgttcgact

SEQ ID NO: 92 - DNA, 18 bp, Artificial Sequence
  Probe for the detection of L. brevis
agcacgtcattcaacgga

SEQ ID NO: 93 - DNA, 18 bp, Artificial Sequence
  Probe for the detection of L. brevis
tctgggcagatttcccac

SEQ ID NO: 94 - DNA, 18 bp, Artificial Sequence
  Probe for the detection of L. brevis
cggaagctcgttcgactt

SEQ ID NO: 95 - DNA, 18 bp, Artificial Sequence
  Probe for the detection of L. brevis
aagcacgtcattcaacgg

SEQ ID NO: 96 - DNA, 18 bp, Artificial Sequence
  Probe for the detection of L. brevis
gttcgccactcgcttcat

SEQ ID NO: 97 - DNA, 18 bp, Artificial Sequence
  Probe for the detection of L. brevis
ccctgcttctgggcagat

SEQ ID NO: 98 - DNA, 18 bp, Artificial Sequence
  Probe for the detection of L. brevis
ctgactcccgaaggttat

SEQ ID NO: 99 - DNA, 18 bp, Artificial Sequence
  Probe for the detection of L. brevis
tgcttctgggcagatttc

SEQ ID NO: 100 - DNA, 18 bp, Artificial Sequence
  Probe for the detection of L. brevis
ttctgggcagatttccca

SEQ ID NO: 101 - DNA, 18 bp, Artificial Sequence
  Probe for the detection of L. brevis
actcccgaaggttatctc

SEQ ID NO: 102 - DNA, 18 bp, Artificial Sequence
  Probe for the detection of L. brevis
cttctgggcagatttccc

SEQ ID NO: 103 - DNA, 18 bp, Artificial Sequence
  Probe for the detection of L. brevis
ctgggcagatttcccacg

SEQ ID NO: 104 - DNA, 18 bp, Artificial Sequence
  Probe for the detection of L. brevis
actaatacgccgcgggat

SEQ ID NO: 105 - DNA, 18 bp, Artificial Sequence
  Probe for the detection of L. brevis
gtgcaagcacgtcattca

SEQ ID NO: 106 - DNA, 18 bp, Artificial Sequence
  Probe for the detection of L. brevis
acggctgactcccgaagg

SEQ ID NO: 107 - DNA, 18 bp, Artificial Sequence
  Probe for the detection of L. brevis
ttagacggctgactcccg

SEQ ID NO: 108 - DNA, 18 bp, Artificial Sequence
  Probe for the detection of L. lindner
gtcacaccgtgagcagtt

SEQ ID NO: 109 - DNA, 18 bp, Artificial Sequence
  Probe for the detection of L. lindner
cgtcacaccgtgagcagt

SEQ ID NO: 110 - DNA, 18 bp, Artificial Sequence
  Probe for the detection of L. lindner
ccactcggtcagatctat

SEQ ID NO: 111 - DNA, 18 bp, Artificial Sequence
  Probe for the detection of L. lindner
gatgcaagcaccagctat

SEQ ID NO: 112 - DNA, 18 bp, Artificial Sequence
  Probe for the detection of L. lindner
tcggtcagatctatcgtc

SEQ ID NO: 113 - DNA, 18 bp, Artificial Sequence
  Probe for the detection of L. lindner
cggtcagatctatcgtca

SEQ ID NO: 114 - DNA, 18 bp, Artificial Sequence
  Probe for the detection of L. lindner
ctcggtcagatctatcgt

SEQ ID NO: 115 - DNA, 18 bp, Artificial Sequence
  Probe for the detection of L. lindner
tcacaccgtgagcagttg

SEQ ID NO: 116 - DNA, 18 bp, Artificial Sequence
  Probe for the detection of L. lindner
ccgtcacaccgtgagcag

SEQ ID NO: 117 - DNA, 18 bp, Artificial Sequence
  Probe for the detection of L. lindner
ctgatgcaagcaccagct

SEQ ID NO: 118 - DNA, 18 bp, Artificial Sequence
  Probe for the detection of L. lindner
cggcggactccgtaaagg

SEQ ID NO: 119 - DNA, 18 bp, Artificial Sequence
  Probe for the detection of L. lindner
gctgatgcaagcaccagc

SEQ ID NO: 120 - DNA, 18 bp, Artificial Sequence
  Probe for the detection of L. lindner
accgtcacaccgtgagca

SEQ ID NO: 121 - DNA, 18 bp, Artificial Sequence
  Probe for the detection of L. lindner
cagatgcagaccagacag

SEQ ID NO: 122 - DNA, 18 bp, Artificial Sequence
  Probe for the detection of L. lindner
tgatgcaagcaccagcta

SEQ ID NO: 123 - DNA, 18 bp, Artificial Sequence
  Probe for the detection of L. lindner
agttaggagacctcgttc

SEQ ID NO: 124 - DNA, 18 bp, Artificial Sequence
  Probe for the detection of L. lindner
ggcggactccgtaaaggt

SEQ ID NO: 125 - DNA, 18 bp, Artificial Sequence
  Probe for the detection of L. lindner
gttaggagacctcgttcg

SEQ ID NO: 126 - DNA, 18 bp, Artificial Sequence
  Probe for the detection of L. lindner
agttgctctcacggtcgt

SEQ ID NO: 127 - DNA, 18 bp, Artificial Sequence
  Probe for the detection of L. lindner
gcaccagctatcagttag

SEQ ID NO: 128 - DNA, 18 bp, Artificial Sequence
  Probe for the detection of L. lindner
taccgtcacaccgtgagc

SEQ ID NO: 129 - DNA, 18 bp, Artificial Sequence
  Probe for the detection of L. lindner
agataccgtcacaccgtg

SEQ ID NO: 130 - DNA, 18 bp, Artificial Sequence
  Probe for the detection of L. lindner
tagataccgtcacaccgt

SEQ ID NO: 131 - DNA, 18 bp, Artificial Sequence
  Probe for the detection of L. lindner
tgctctcacggtcgttct

SEQ ID NO: 132 - DNA, 18 bp, Artificial Sequence
  Probe for the detection of L. lindner
accatgtggttctcgttg

SEQ ID NO: 133 - DNA, 18 bp, Artificial Sequence
  Probe for the detection of L. lindner
atgcaagcaccagctatc

SEQ ID NO: 134 - DNA, 18 bp, Artificial Sequence
  Probe for the detection of L. lindner
ggcggcggactccgtaaa

SEQ ID NO: 135 - DNA, 18 bp, Artificial Sequence
  Probe for the detection of L. lindner
aggcggcggactccgtaa

SEQ ID NO: 136 - DNA, 18 bp, Artificial Sequence
  Probe for the detection of L. lindner
cacaccgtgagcagttgc

SEQ ID NO: 137 - DNA, 18 bp, Artificial Sequence
  Probe for the detection of L. lindner
ttagataccgtcacaccg

SEQ ID NO: 138 - DNA, 18 bp, Artificial Sequence
  Probe for the detection of L. lindner
gaaccatgtggttctcgt

SEQ ID NO: 139 - DNA, 18 bp, Artificial Sequence
  Probe for the detection of L. lindner
gctctcacggtcgttctt

SEQ ID NO: 140 - DNA, 18 bp, Artificial Sequence
  Probe for the detection of L. lindner
caccagctatcagttagg

SEQ ID NO: 141 - DNA, 18 bp, Artificial Sequence
  Probe for the detection of L. lindner
gccactcggtcagatcta

SEQ ID NO: 142 - DNA, 18 bp, Artificial Sequence
  Probe for the detection of L. lindner
gataccgtcacaccgtga

SEQ ID NO: 143 - DNA, 18 bp, Artificial Sequence
  Probe for the detection of L. lindner
tcagatgcagaccagaca

SEQ ID NO: 144 - DNA, 18 bp, Artificial Sequence
  Probe for the detection of L. lindner
taggcggcggactccgta

SEQ ID NO: 145 - DNA, 18 bp, Artificial Sequence
  Probe for the detection of L. lindner
ccatgtggttctcgttgt

SEQ ID NO: 146 - DNA, 18 bp, Artificial Sequence
  Probe for the detection of L. lindner
caagcaccagctatcagt

SEQ ID NO: 147 - DNA, 18 bp, Artificial Sequence
  Probe for the detection of L. buchneri
cgctgagtggcgcgggtt

SEQ ID NO: 148 - DNA, 18 bp, Artificial Sequence
  Probe for the detection of L. buchneri
ccggattccgacgacgtt

SEQ ID NO: 149 - DNA, 18 bp, Artificial Sequence
  Probe for the detection of L. buchneri
cgccaaccttcccagatt

SEQ ID NO: 150 - DNA, 18 bp, Artificial Sequence
  Probe for the detection of L. buchneri
acgacgtttcacgtgtgt

SEQ ID NO: 151 - DNA, 18 bp, Artificial Sequence
  Probe for the detection of L. buchneri
cgacgacgtttcacgtgt

SEQ ID NO: 152 - DNA, 18 bp, Artificial Sequence
  Probe for the detection of L. buchneri
caagtccacagtctcggt

SEQ ID NO: 153 - DNA, 18 bp, Artificial Sequence
  Probe for the detection of L. buchneri
ctacccagcggtggcggt

SEQ ID NO: 154 - DNA, 18 bp, Artificial Sequence
  Probe for the detection of L. buchneri
aacctggcatgttaccgt

SEQ ID NO: 155 - DNA, 18 bp, Artificial Sequence
  Probe for the detection of L. buchneri
gcgcacagcaccccttct

SEQ ID NO: 156 - DNA, 18 bp, Artificial Sequence
  Probe for the detection of L. buchneri
accagtccttaacggtct

SEQ ID NO: 157 - DNA, 18 bp, Artificial Sequence
  Probe for the detection of L. buchneri
aggtcaagtccacagtct

SEQ ID NO: 158 - DNA, 18 bp, Artificial Sequence
  Probe for the detection of L. buchneri
ttccccacgtctacctct

SEQ ID NO: 159 - DNA, 18 bp, Artificial Sequence
  Probe for the detection of L. buchneri
tccactcccaacctatct

SEQ ID NO: 160 - DNA, 18 bp, Artificial Sequence
  Probe for the detection of L. buchneri
gggcttcatttctgggct

SEQ ID NO: 161 - DNA, 18 bp, Artificial Sequence
  Probe for the detection of L. buchneri
gattctacgtccgaggct

SEQ ID NO: 162 - DNA, 18 bp, Artificial Sequence
  Probe for the detection of L. buchneri
tgcacaacttagcctcct

SEQ ID NO: 163 - DNA, 18 bp, Artificial Sequence
  Probe for the detection of L. buchneri
cttgcgcacagcacccct

SEQ ID NO: 164 - DNA, 18 bp, Artificial Sequence
  Probe for the detection of L. buchneri
agttccccacgtctacct

SEQ ID NO: 165 - DNA, 18 bp, Artificial Sequence
  Probe for the detection of L. buchneri
gctccggcttttaaacct

SEQ ID NO: 166 - DNA, 18 bp, Artificial Sequence
  Probe for the detection of L. buchneri
agcctccccaggaaacct

SEQ ID NO: 167 - DNA, 18 bp, Artificial Sequence
  Probe for the detection of L. buchneri
gttggttgcttccctact

SEQ ID NO: 168 - DNA, 18 bp, Artificial Sequence
  Probe for the detection of L. buchneri
ggcggtggcggcgcaact

SEQ ID NO: 169 - DNA, 18 bp, Artificial Sequence
  Probe for the detection of L. buchneri
ccccacgtctacctctat

SEQ ID NO: 170 - DNA, 18 bp, Artificial Sequence
  Probe for the detection of L. buchneri
cttccactcccaacctat

SEQ ID NO: 171 - DNA, 18 bp, Artificial Sequence
  Probe for the detection of L. buchneri
tcgccaaccttcccagat

SEQ ID NO: 172 - DNA, 18 bp, Artificial Sequence
  Probe for the detection of L. buchneri
ttggtccgctccgtacat

SEQ ID NO: 173 - DNA, 18 bp, Artificial Sequence
  Probe for the detection of L. buchneri
gctgtgtcaacacccaat

SEQ ID NO: 174 - DNA, 18 bp, Artificial Sequence
  Probe for the detection of L. buchneri
gccaaccttcccagattg

SEQ ID NO: 175 - DNA, 18 bp, Artificial Sequence
  Probe for the detection of L. buchneri
gacgacgtttcacgtgtg

SEQ ID NO: 176 - DNA, 18 bp, Artificial Sequence
  Probe for the detection of L. buchneri
tacccagcggtggcggtg

SEQ ID NO: 177 - DNA, 18 bp, Artificial Sequence
  Probe for the detection of L. buchneri
gcacaacttagcctcctg

SEQ ID NO: 178 - DNA, 18 bp, Artificial Sequence
  Probe for the detection of L. buchneri
gcggtggcggcgcaactg

SEQ ID NO: 179 - DNA, 18 bp, Artificial Sequence
  Probe for the detection of L. buchneri
acccagcggtggcggtgg

SEQ ID NO: 180 - DNA, 18 bp, Artificial Sequence
  Probe for the detection of L. buchneri
cggtggcggcgcaactgg

SEQ ID NO: 181 - DNA, 18 bp, Artificial Sequence
  Probe for the detection of L. buchneri
ttgatttcacctacgggg

SEQ ID NO: 182 - DNA, 18 bp, Artificial Sequence
  Probe for the detection of L. buchneri
cacgctgagtggcgcggg

SEQ ID NO: 183 - DNA, 18 bp, Artificial Sequence
  Probe for the detection of L. buchneri
aggatcctgaactgaggg

SEQ ID NO: 184 - DNA, 18 bp, Artificial Sequence
  Probe for the detection of L. buchneri
tcaagtccacagtctcgg

SEQ ID NO: 185 - DNA, 18 bp, Artificial Sequence
  Probe for the detection of L. buchneri
cagcggtggcggtggcgg

SEQ ID NO: 186 - DNA, 18 bp, Artificial Sequence
  Probe for the detection of L. buchneri
ccacgctgagtggcgcgg

SEQ ID NO: 187 - DNA, 18 bp, Artificial Sequence
  Probe for the detection of L. buchneri
tccatacggtaccaccgg

SEQ ID NO: 188 - DNA, 18 bp, Artificial Sequence
  Probe for the detection of L. casei
ccgtcacgccgacaacag

SEQ ID NO: 189 - DNA, 18 bp, Artificial Sequence
  Probe for the detection of L. casei
accgtcacgccgacaaca

SEQ ID NO: 190 - DNA, 18 bp, Artificial Sequence
  Probe for the detection of L. casei
ataccgtcacgccgacaa

SEQ ID NO: 191 - DNA, 18 bp, Artificial Sequence
  Probe for the detection of L. casei
taccgtcacgccgacaac

SEQ ID NO: 192 - DNA, 18 bp, Artificial Sequence
  Probe for the detection of L. casei
gataccgtcacgccgaca

SEQ ID NO: 193 - DNA, 18 bp, Artificial Sequence
  Probe for the detection of L. casei
ggataccgtcacgccgac

SEQ ID NO: 194 - DNA, 18 bp, Artificial Sequence
  Probe for the detection of L. casei
acgccgacaacagttact

SEQ ID NO: 195 - DNA, 18 bp, Artificial Sequence
  Probe for the detection of L. casei
ggctcgctccctaaaagg

SEQ ID NO: 196 - DNA, 18 bp, Artificial Sequence
  Probe for the detection of L. casei
ctctgccgaccattcttc

SEQ ID NO: 197 - DNA, 18 bp, Artificial Sequence
  Probe for the detection of L. casei
ctgccgaccattcttctc

SEQ ID NO: 198 - DNA, 18 bp, Artificial Sequence
  Probe for the detection of L. casei
cgccgacaacagttactc

SEQ ID NO: 199 - DNA, 18 bp, Artificial Sequence
  Probe for the detection of L. casei
cacgccgacaacagttac

SEQ ID NO: 200 - DNA, 18 bp, Artificial Sequence
  Probe for the detection of L. casei
tcacgccgacaacagtta

SEQ ID NO: 201 - DNA, 18 bp, Artificial Sequence
  Probe for the detection of L. casei
tctgccgaccattcttct

SEQ ID NO: 202 - DNA, 18 bp, Artificial Sequence
  Probe for the detection of L. casei
acaacagttactctgccg

SEQ ID NO: 203 - DNA, 18 bp, Artificial Sequence
  Probe for the detection of L. casei
cggctcgctccctaaaag

SEQ ID NO: 204 - DNA, 18 bp, Artificial Sequence
  Probe for the detection of L. casei
gacaacagttactctgcc

SEQ ID NO: 205 - DNA, 18 bp, Artificial Sequence
  Probe for the detection of L. casei
acggctcgctccctaaaa

SEQ ID NO: 206 - DNA, 18 bp, Artificial Sequence
  Probe for the detection of L. casei
cgacaacagttactctgc

SEQ ID NO: 207 - DNA, 18 bp, Artificial Sequence
  Probe for the detection of L. casei
ccgacaacagttactctg

SEQ ID NO: 208 - DNA, 18 bp, Artificial Sequence
  Probe for the detection of L. casei
actctgccgaccattctt

SEQ ID NO: 209 - DNA, 18 bp, Artificial Sequence
  Probe for the detection of L. casei
ctcgctccctaaaagggt

SEQ ID NO: 210 - DNA, 18 bp, Artificial Sequence
  Probe for the detection of L. casei
tgccgaccattcttctcc

SEQ ID NO: 211 - DNA, 18 bp, Artificial Sequence
  Probe for the detection of L. casei
gccgaccattcttctcca

SEQ ID NO: 212 - DNA, 18 bp, Artificial Sequence
  Probe for the detection of L. casei
cgccatctttcagccaag

SEQ ID NO: 213 - DNA, 18 bp, Artificial Sequence
  Probe for the detection of L. casei
gacggctcgctccctaaa

SEQ ID NO: 214 - DNA, 18 bp, Artificial Sequence
  Probe for the detection of L. casei
cgaccattcttctccaac

SEQ ID NO: 215 - DNA, 18 bp, Artificial Sequence
  Probe for the detection of L. casei
gtcacgccgacaacagtt

SEQ ID NO: 216 - DNA, 18 bp, Artificial Sequence
  Probe for the detection of L. casei
cctgatctctcaggtgat

SEQ ID NO: 217 - DNA, 18 bp, Artificial Sequence
  Probe for the detection of L. casei
aacagttactctgccgac

SEQ ID NO: 218 - DNA, 18 bp, Artificial Sequence
  Probe for the detection of L. casei
tactctgccgaccattct

SEQ ID NO: 219 - DNA, 18 bp, Artificial Sequence
  Probe for the detection of L. casei
ccgaccattcttctccaa

SEQ ID NO: 220 - DNA, 18 bp, Artificial Sequence
  Probe for the detection of L. casei
gccgacaacagttactct

SEQ ID NO: 221 - DNA, 18 bp, Artificial Sequence
  Probe for the detection of L. casei
ttactctgccgaccattc

SEQ ID NO: 222 - DNA, 18 bp, Artificial Sequence
  Probe for the detection of L. casei
tccctaaaagggttacgc

SEQ ID NO: 223 - DNA, 18 bp, Artificial Sequence
  Probe for the detection of L. casei
caacagttactctgccga

SEQ ID NO: 224 - DNA, 18 bp, Artificial Sequence
  Probe for the detection of L. casei
agacggctcgctccctaa

SEQ ID NO: 225 - DNA, 18 bp, Artificial Sequence
  Probe for the detection of L. casei
acgccatctttcagccaa

SEQ ID NO: 226 - DNA, 18 bp, Artificial Sequence
  Probe for the detection of L. casei
aacctgatctctcaggtg

SEQ ID NO: 227 - DNA, 18 bp, Artificial Sequence
  Probe for the detection of L. coryniformis
actgcaagcagcttcggt

SEQ ID NO: 228 - DNA, 18 bp, Artificial Sequence
  Probe for the detection of L. coryniformis
cgtccactgcaagcagct

SEQ ID NO: 229 - DNA, 18 bp, Artificial Sequence
  Probe for the detection of L. coryniformis
gtcaatcaacgtccactg

SEQ ID NO: 230 - DNA, 18 bp, Artificial Sequence
  Probe for the detection of L. coryniformis
cactgcaagcagcttcgg

SEQ ID NO: 231 - DNA, 18 bp, Artificial Sequence
  Probe for the detection of L. coryniformis
gtctgaatggttatgcgg

SEQ ID NO: 232 - DNA, 18 bp, Artificial Sequence
  Probe for the detection of L. coryniformis
tcgacgtcagtgcgttcg

SEQ ID NO: 233 - DNA, 18 bp, Artificial Sequence
  Probe for the detection of L. coryniformis
ccactgcaagcagcttcg

SEQ ID NO: 234 - DNA, 18 bp, Artificial Sequence
  Probe for the detection of L. coryniformis
tgcaagcagcttcggtcg

SEQ ID NO: 235 - DNA, 18 bp, Artificial Sequence
  Probe for the detection of L. coryniformis
aagcagcttcggtcgacg

SEQ ID NO: 236 - DNA, 18 bp, Artificial Sequence
  Probe for the detection of L. coryniformis
aacgtccactgcaagcag

SEQ ID NO: 237 - DNA, 18 bp, Artificial Sequence
  Probe for the detection of L. coryniformis
gtcgacgtcagtgcgttc

SEQ ID NO: 238 - DNA, 18 bp, Artificial Sequence
  Probe for the detection of L. coryniformis
tccactgcaagcagcttc

SEQ ID NO: 239 - DNA, 18 bp, Artificial Sequence
  Probe for the detection of L. coryniformis
gcagcttcggtcgacgtc

SEQ ID NO: 240 - DNA, 18 bp, Artificial Sequence
  Probe for the detection of L. coryniformis
tcaatcaacgtccactgc

SEQ ID NO: 241 - DNA, 18 bp, Artificial Sequence
  Probe for the detection of L. coryniformis
acgtccactgcaagcagc

SEQ ID NO: 242 - DNA, 18 bp, Artificial Sequence
  Probe for the detection of L. coryniformis
tcaacgtccactgcaagc

SEQ ID NO: 243 - DNA, 18 bp, Artificial Sequence
  Probe for the detection of L. coryniformis
caagcagcttcggtcgac

SEQ ID NO: 244 - DNA, 18 bp, Artificial Sequence
  Probe for the detection of L. coryniformis
cgacgtcagtgcgttcga

SEQ ID NO: 245 - DNA, 18 bp, Artificial Sequence
  Probe for the detection of L. coryniformis
gcaagcagcttcggtcga

SEQ ID NO: 246 - DNA, 18 bp, Artificial Sequence
  Probe for the detection of L. coryniformis
cagcttcggtcgacgtca

SEQ ID NO: 247 - DNA, 18 bp, Artificial Sequence
  Probe for the detection of L. coryniformis
caatcaacgtccactgca

SEQ ID NO: 248 - DNA, 18 bp, Artificial Sequence
  Probe for the detection of L. coryniformis
caacgtccactgcaagca

SEQ ID NO: 249 - DNA, 18 bp, Artificial Sequence
  Probe for the detection of L. coryniformis
gacgtcagtgcgttcgac

SEQ ID NO: 250 - DNA, 18 bp, Artificial Sequence
  Probe for the detection of L. coryniformis
gtccactgcaagcagctt

SEQ ID NO: 251 - DNA, 18 bp, Artificial Sequence
  Probe for the detection of L. coryniformis
atcaacgtccactgcaag

SEQ ID NO: 252 - DNA, 18 bp, Artificial Sequence
  Probe for the detection of L. coryniformis
ccgtcaaaggactaacag

SEQ ID NO: 253 - DNA, 18 bp, Artificial Sequence
  Probe for the detection of L. coryniformis
ggtctgaatggttatgcg

SEQ ID NO: 254 - DNA, 18 bp, Artificial Sequence
  Probe for the detection of L. coryniformis
cgtcaatcaacgtccact

SEQ ID NO: 255 - DNA, 18 bp, Artificial Sequence
  Probe for the detection of L. coryniformis
cagttactctagtccctg

SEQ ID NO: 256 - DNA, 18 bp, Artificial Sequence
  Probe for the detection of L. coryniformis
agcttcggtcgacgtcag

SEQ ID NO: 257 - DNA, 18 bp, Artificial Sequence
  Probe for the detection of L. coryniformis
ctgcaagcagcttcggtc

SEQ ID NO: 258 - DNA, 18 bp, Artificial Sequence
  Probe for the detection of L. coryniformis
ctagtccctgttcttctc

SEQ ID NO: 259 - DNA, 18 bp, Artificial Sequence
  Probe for the detection of L. coryniformis
ggataccgtcaaaggact

SEQ ID NO: 260 - DNA, 18 bp, Artificial Sequence
  Probe for the detection of L. coryniformis
agcagcttcggtcgacgt

SEQ ID NO: 261 - DNA, 18 bp, Artificial Sequence
  Probe for the detection of L. coryniformis
acgtcaatcaacgtccac

SEQ ID NO: 262 - DNA, 18 bp, Artificial Sequence
  Probe for the detection of L. coryniformis
gcttcggtcgacgtcagt

SEQ ID NO: 263 - DNA, 18 bp, Artificial Sequence
  Probe for the detection of L. coryniformis
acgtcagtgcgttcgact

SEQ ID NO: 264 - DNA, 18 bp, Artificial Sequence
  Probe for the detection of L. coryniformis
accatgtggtctgaatgg

SEQ ID NO: 265 - DNA, 18 bp, Artificial Sequence
  Probe for the detection of L. coryniformis
tccctaaaagggttaccc

SEQ ID NO: 266 - DNA, 18 bp, Artificial Sequence
  Probe for the detection of L. fructivorans
ctatcattaggcgcagct

SEQ ID NO: 267 - DNA, 18 bp, Artificial Sequence
  Probe for the detection of L. fructivorans
actatcattaggcgcagc

SEQ ID NO: 268 - DNA, 18 bp, Artificial Sequence
  Probe for the detection of L. fructivorans
ggcgcagctcgttcgact

SEQ ID NO: 269 - DNA, 18 bp, Artificial Sequence
  Probe for the detection of L. fructivorans
gcggcaggctccaaaagg

SEQ ID NO: 270 - DNA, 18 bp, Artificial Sequence
  Probe for the detection of L. fructivorans
attaggcgcagctcgttc

SEQ ID NO: 271 - DNA, 18 bp, Artificial Sequence
  Probe for the detection of L. fructivorans
atcattaggcgcagctcg

SEQ ID NO: 272 - DNA, 18 bp, Artificial Sequence
  Probe for the detection of L. fructivorans
aggcgcagctcgttcgac

SEQ ID NO: 273 - DNA, 18 bp, Artificial Sequence
  Probe for the detection of L. fructivorans
cggcaggctccaaaaggt

SEQ ID NO: 274 - DNA, 18 bp, Artificial Sequence
  Probe for the detection of L. fructivorans
tcattaggcgcagctcgt

SEQ ID NO: 275 - DNA, 18 bp, Artificial Sequence
  Probe for the detection of L. fructivorans
gcgcagctcgttcgactt

SEQ ID NO: 276 - DNA, 18 bp, Artificial Sequence
  Probe for the detection of L. fructivorans
ttaggcgcagctcgttcg

SEQ ID NO: 277 - DNA, 18 bp, Artificial Sequence
  Probe for the detection of L. fructivorans
ggcaggctccaaaaggtt

SEQ ID NO: 278 - DNA, 18 bp, Artificial Sequence
  Probe for the detection of L. fructivorans
tatcattaggcgcagctc

SEQ ID NO: 279 - DNA, 18 bp, Artificial Sequence
  Probe for the detection of L. fructivorans
gcaggctccaaaaggtta

SEQ ID NO: 280 - DNA, 18 bp, Artificial Sequence
  Probe for the detection of L. fructivorans
cgcagctcgttcgacttg

SEQ ID NO: 281 - DNA, 18 bp, Artificial Sequence
  Probe for the detection of L. fructivorans
cattaggcgcagctcgtt

SEQ ID NO: 282 - DNA, 18 bp, Artificial Sequence
  Probe for the detection of L. fructivorans
taggcgcagctcgttcga

SEQ ID NO: 283 - DNA, 18 bp, Artificial Sequence
  Probe for the detection of L. fructivorans
tagataccgtcgcgacgt

SEQ ID NO: 284 - DNA, 18 bp, Artificial Sequence
  Probe for the detection of L. fructivorans
ttagataccgtcgcgacg

SEQ ID NO: 285 - DNA, 18 bp, Artificial Sequence
  Probe for the detection of L. fructivorans
ataccgtcgcgacgtgag

SEQ ID NO: 286 - DNA, 18 bp, Artificial Sequence
  Probe for the detection of L. fructivorans
taccgtcgcgacgtgagc

SEQ ID NO: 287 - DNA, 18 bp, Artificial Sequence
  Probe for the detection of L. fructivorans
gttagataccgtcgcgac

SEQ ID NO: 288 - DNA, 18 bp, Artificial Sequence
  Probe for the detection of L. fructivorans
gataccgtcgcgacgtga

SEQ ID NO: 289 - DNA, 18 bp, Artificial Sequence
  Probe for the detection of L. fructivorans
accgtcgcgacgtgagca

SEQ ID NO: 290 - DNA, 18 bp, Artificial Sequence
  Probe for the detection of L. fructivorans
caggctccaaaaggttac

SEQ ID NO: 291 - DNA, 18 bp, Artificial Sequence
  Probe for the detection of L. fructivorans
cacgcccgttcttctcta

SEQ ID NO: 292 - DNA, 18 bp, Artificial Sequence
  Probe for the detection of L. fructivorans
gcgacgtgagcagttact

SEQ ID NO: 293 - DNA, 18 bp, Artificial Sequence
  Probe for the detection of L. fructivorans
cgcgacgtgagcagttac

SEQ ID NO: 294 - DNA, 18 bp, Artificial Sequence
  Probe for the detection of L. fructivorans
gcacaaaggccatctttc

SEQ ID NO: 295 - DNA, 18 bp, Artificial Sequence
  Probe for the detection of L. fructivorans
aggcggcaggctccaaaa

SEQ ID NO: 296 - DNA, 18 bp, Artificial Sequence
  Probe for the detection of L. fructivorans
agttactctcacgcccgt

SEQ ID NO: 297 - DNA, 18 bp, Artificial Sequence
  Probe for the detection of L. fructivorans
gatagcacaaaggccatc

SEQ ID NO: 298 - DNA, 18 bp, Artificial Sequence
  Probe for the detection of L. fructivorans
tcgcgacgtgagcagtta

SEQ ID NO: 299 - DNA, 18 bp, Artificial Sequence
  Probe for the detection of L. fructivorans
ccaccttaggcggcaggc

SEQ ID NO: 300 - DNA, 18 bp, Artificial Sequence
  Probe for the detection of L. fructivorans
accttaggcggcaggctc

SEQ ID NO: 301 - DNA, 18 bp, Artificial Sequence
  Probe for the detection of L. fructivorans
taggcggcaggctccaaa

SEQ ID NO: 302 - DNA, 18 bp, Artificial Sequence
  Probe for the detection of L. fructivorans
gcagttactctcacgccc

SEQ ID NO: 303 - DNA, 18 bp, Artificial Sequence
  Probe for the detection of L. fructivorans
cgacgtgagcagttactc

SEQ ID NO: 304 - DNA, 18 bp, Artificial Sequence
  Probe for the detection of L. fructivorans
cagttactctcacgcccg

SEQ ID NO: 305 - DNA, 18 bp, Artificial Sequence
  Probe for the detection of Lactobacillus perolens
ccatgcggtctccgtggt

SEQ ID NO: 306 - DNA, 18 bp, Artificial Sequence
  Probe for the detection of Lactobacillus perolens
catgcggtctccgtggtt

SEQ ID NO: 307 - DNA, 18 bp, Artificial Sequence
  Probe for the detection of Lactobacillus perolens
tgcggtctccgtggttat

SEQ ID NO: 308 - DNA, 18 bp, Artificial Sequence
  Probe for the detection of Lactobacillus perolens
gaccatgcggtctccgtg

SEQ ID NO: 309 - DNA, 18 bp, Artificial Sequence
  Probe for the detection of Lactobacillus perolens
ggtgatgcaagcaccacc

SEQ ID NO: 310 - DNA, 18 bp, Artificial Sequence
  Probe for the detection of Lactobacillus perolens
catcttttacccggagac

SEQ ID NO: 311 - DNA, 18 bp, Artificial Sequence
  Probe for the detection of Lactobacillus perolens
atgcggtctccgtggtta

SEQ ID NO: 312 - DNA, 18 bp, Artificial Sequence
  Probe for the detection of Lactobacillus perolens
agaccatgcggtctccgt

SEQ ID NO: 313 - DNA, 18 bp, Artificial Sequence
  Probe for the detection of Lactobacillus perolens
gtgatgcaagcaccaccg

SEQ ID NO: 314 - DNA, 18 bp, Artificial Sequence
  Probe for the detection of Lactobacillus perolens
attggtgatgcaagcacc

SEQ ID NO: 315 - DNA, 18 bp, Artificial Sequence
  Probe for the detection of Lactobacillus perolens
tgatgcaagcaccaccgc

SEQ ID NO: 316 - DNA, 18 bp, Artificial Sequence
  Probe for the detection of Lactobacillus perolens
accatgcggtctccgtgg

SEQ ID NO: 317 - DNA, 18 bp, Artificial Sequence
  Probe for the detection of Lactobacillus perolens
ttggtgatgcaagcacca

SEQ ID NO: 318 - DNA, 18 bp, Artificial Sequence
  Probe for the detection of Lactobacillus perolens
cttttacccggagaccat

SEQ ID NO: 319 - DNA, 18 bp, Artificial Sequence
  Probe for the detection of Lactobacillus perolens
atcttttacccggagacc

SEQ ID NO: 320 - DNA, 18 bp, Artificial Sequence
  Probe for the detection of Lactobacillus perolens
ttacccggagaccatgcg

SEQ ID NO: 321 - DNA, 18 bp, Artificial Sequence
  Probe for the detection of Lactobacillus perolens
tcttttacccggagacca

SEQ ID NO: 322 - DNA, 18 bp, Artificial Sequence
  Probe for the detection of Lactobacillus perolens
ggtctccgtggttatacg

SEQ ID NO: 323 - DNA, 18 bp, Artificial Sequence
  Probe for the detection of Lactobacillus perolens
gatgcaagcaccaccgca

SEQ ID NO: 324 - DNA, 18 bp, Artificial Sequence
  Probe for the detection of Lactobacillus perolens
gagaccatgcggtctccg

SEQ ID NO: 325 - DNA, 18 bp, Artificial Sequence
  Probe for the detection of Lactobacillus perolens
cggtctccgtggttatac

SEQ ID NO: 326 - DNA, 18 bp, Artificial Sequence
  Probe for the detection of Lactobacillus perolens
tttacccggagaccatgc

SEQ ID NO: 327 - DNA, 18 bp, Artificial Sequence
  Probe for the detection of Lactobacillus perolens
tgcaagcaccaccgcaaa

SEQ ID NO: 328 - DNA, 18 bp, Artificial Sequence
  Probe for the detection of Lactobacillus perolens
ggagaccatgcggtctcc

SEQ ID NO: 329 - DNA, 18 bp, Artificial Sequence
  Probe for the detection of Lactobacillus perolens
gcaagcaccaccgcaaac

SEQ ID NO: 330 - DNA, 18 bp, Artificial Sequence
  Probe for the detection of Lactobacillus perolens
ttttacccggagaccatg

SEQ ID NO: 331 - DNA, 18 bp, Artificial Sequence
  Probe for the detection of Lactobacillus perolens
agcaccaccgcaaactga

SEQ ID NO: 332 - DNA, 18 bp, Artificial Sequence
  Probe for the detection of Lactobacillus perolens
aagcaccaccgcaaactg

SEQ ID NO: 333 - DNA, 18 bp, Artificial Sequence
  Probe for the detection of Lactobacillus perolens
ccatcttttacccggaga

SEQ ID NO: 334 - DNA, 18 bp, Artificial Sequence
  Probe for the detection of Lactobacillus perolens
atgcaagcaccaccgcaa

SEQ ID NO: 335 - DNA, 18 bp, Artificial Sequence
  Probe for the detection of Lactobacillus perolens
gtctccgtggttatacgg

SEQ ID NO: 336 - DNA, 18 bp, Artificial Sequence
  Probe for the detection of Lactobacillus perolens
gcggtctccgtggttata

SEQ ID NO: 337 - DNA, 18 bp, Artificial Sequence
  Probe for the detection of Lactobacillus perolens
caagcaccaccgcaaact

SEQ ID NO: 338 - DNA, 18 bp, Artificial Sequence
  Probe for the detection of Lactobacillus perolens
tctccgtggttatacggt

SEQ ID NO: 339 - DNA, 18 bp, Artificial Sequence
  Probe for the detection of Lactobacillus perolens
ctccgtggttatacggta

SEQ ID NO: 340 - DNA, 18 bp, Artificial Sequence
  Probe for the detection of Lactobacillus perolens
gccatcttttacccggag

SEQ ID NO: 341 - DNA, 18 bp, Artificial Sequence
  Probe for the detection of Lactobacillus perolens
cgccatcttttacccgga

SEQ ID NO: 342 - DNA, 18 bp, Artificial Sequence
  Probe for the detection of Lactobacillus perolens
cagctgatctctcagcct

SEQ ID NO: 343 - DNA, 18 bp, Artificial Sequence
  Probe for the detection of Lactobacillus perolens
cgcaaactgaccaaacct

SEQ ID NO: 344 - DNA, 18 bp, Artificial Sequence
  Probe for the detection of Lactobacillus plantarum
aagctcggaccatgcggt

SEQ ID NO: 345 - DNA, 18 bp, Artificial Sequence
  Probe for the detection of Lactobacillus plantarum
ctttcaagctcggaccat

SEQ ID NO: 346 - DNA, 18 bp, Artificial Sequence
  Probe for the detection of Lactobacillus plantarum
tttcaagctcggaccatg

SEQ ID NO: 347 - DNA, 18 bp, Artificial Sequence
  Probe for the detection of Lactobacillus plantarum
gccatctttcaagctcgg

SEQ ID NO: 348 - DNA, 18 bp, Artificial Sequence
  Probe for the detection of Lactobacillus plantarum
caagctcggaccatgcgg

SEQ ID NO: 349 - DNA, 18 bp, Artificial Sequence
  Probe for the detection of Lactobacillus plantarum
agccatctttcaagctcg

SEQ ID NO: 350 - DNA, 18 bp, Artificial Sequence
  Probe for the detection of Lactobacillus plantarum
tcaagctcggaccatgcg

SEQ ID NO: 351 - DNA, 18 bp, Artificial Sequence
  Probe for the detection of Lactobacillus plantarum
agctcggaccatgcggtc

SEQ ID NO: 352 - DNA, 18 bp, Artificial Sequence
  Probe for the detection of Lactobacillus plantarum
ttcaagctcggaccatgc

SEQ ID NO: 353 - DNA, 18 bp, Artificial Sequence
  Probe for the detection of Lactobacillus plantarum
cgaagccatctttcaagc

SEQ ID NO: 354 - DNA, 18 bp, Artificial Sequence
  Probe for the detection of Lactobacillus plantarum
gctcggaccatgcggtcc

SEQ ID NO: 355 - DNA, 18 bp, Artificial Sequence
  Probe for the detection of Lactobacillus plantarum
atctttcaagctcggacc

SEQ ID NO: 356 - DNA, 18 bp, Artificial Sequence
  Probe for the detection of Lactobacillus plantarum
catctttcaagctcggac

SEQ ID NO: 357 - DNA, 18 bp, Artificial Sequence
  Probe for the detection of Megasphaera cerevisiae
catgcggcctttagatcg

SEQ ID NO: 358 - DNA, 18 bp, Artificial Sequence
  Probe for the detection of Megasphaera cerevisiae
tccgacactccagtccgg

SEQ ID NO: 359 - DNA, 18 bp, Artificial Sequence
  Probe for the detection of Pectinatus cerevisiiphilus
atagtgccgttcgtcccc

SEQ ID NO: 360 - DNA, 18 bp, Artificial Sequence
  Probe for the detection of Pectinatus cerevisiiphilus
ttgctccggcacagaaag

SEQ ID NO: 361 - DNA, 20 bp, Artificial Sequence
  Probe for the detection of bacteria belonging to the genus Pectinatus
gccccttagccggcttcggg

SEQ ID NO: 362 - DNA, 18 bp, Artificial Sequence
  Probe for the detection of bacteria belonging to the genus Pectinatus
gcggcccttagccggctt

SEQ ID NO: 363 - DNA, 18 bp, Artificial Sequence
  Probe for the detection of bacteria belonging to the genus Pectinatus
tgcggcccttagccggct

SEQ ID NO: 364 - DNA, 18 bp, Artificial Sequence
  Probe for the detection of bacteria belonging to the genus Pectinatus
ccttgcggcccttagccg

SEQ ID NO: 365 - DNA, 18 bp, Artificial Sequence
  Probe for the detection of bacteria belonging to the genus Pectinatus
cggcccttagccggcttc

SEQ ID NO: 366 - DNA, 18 bp, Artificial Sequence
  Probe for the detection of bacteria belonging to the genus Pectinatus
ttgcggcccttagccggc

SEQ ID NO: 367 - DNA, 18 bp, Artificial Sequence
  Probe for the detection of bacteria belonging to the genus Pectinatus
tgcgccgttaccgtcacc

SEQ ID NO: 368 - DNA, 18 bp, Artificial Sequence
  Probe for the detection of bacteria belonging to the genus Pectinatus
ggcccttagccggcttcg

SEQ ID NO: 369 - DNA, 18 bp, Artificial Sequence
  Probe for the detection of bacteria belonging to the genus Pectinatus
gcgccgttaccgtcacca

SEQ ID NO: 370 - DNA, 18 bp, Artificial Sequence
  Probe for the detection of bacteria belonging to the genus Pectinatus
cgcacttttaagatccgc

SEQ ID NO: 371 - DNA, 18 bp, Artificial Sequence
  Probe for the detection of bacteria belonging to the genus Pectinatus
gtgcgccgttaccgtcac

SEQ ID NO: 372 - DNA, 18 bp, Artificial Sequence
  Probe for the detection of bacteria belonging to the genus Pectinatus
cgccgttaccgtcaccaa

SEQ ID NO: 373 - DNA, 18 bp, Artificial Sequence
  Probe for the detection of bacteria belonging to the genus Pectinatus
agacggtcggtgccttgc

SEQ ID NO: 374 - DNA, 18 bp, Artificial Sequence
  Probe for the detection of bacteria belonging to the genus Pectinatus
gcccttagccggcttcgg

SEQ ID NO: 375 - DNA, 18 bp, Artificial Sequence
  Probe for the detection of bacteria belonging to the genus Pectinatus
ggtgcgccgttaccgtca

SEQ ID NO: 376 - DNA, 18 bp, Artificial Sequence
  Probe for the detection of bacteria belonging to the genus Pectinatus
gacggtcggtgccttgcg

SEQ ID NO: 377 - DNA, 18 bp, Artificial Sequence
  Probe for the detection of bacteria belonging to the genus Pectinatus
tgccttgcggcccttagc

SEQ ID NO: 378 - DNA, 18 bp, Artificial Sequence
  Probe for the detection of bacteria belonging to the genus Pectinatus
gccttgcggcccttagcc

SEQ ID NO: 379 - DNA, 18 bp, Artificial Sequence
  Probe for the detection of bacteria belonging to the genus Pectinatus
tgacctgcgattagtagc

SEQ ID NO: 380 - DNA, 18 bp, Artificial Sequence
  Probe for the detection of bacteria belonging to the genus Pectinatus
cttgcggcccttagccgg

SEQ ID NO: 381 - DNA, 18 bp, Artificial Sequence
  Probe for the detection of bacteria belonging to the genus Pectinatus
tggtgcgccgttaccgtc

SEQ ID NO: 382 - DNA, 18 bp, Artificial Sequence
  Probe for the detection of bacteria belonging to the genus Pectinatus
gacctgcgattagtagcg

SEQ ID NO: 383 - DNA, 18 bp, Artificial Sequence
  Probe for the detection of bacteria belonging to the genus Pectinatus
ccttagccggcttcgggt

SEQ ID NO: 384 - DNA, 18 bp, Artificial Sequence
  Probe for the detection of bacteria belonging to the genus Pectinatus
ccgcacttttaagatccg

SEQ ID NO: 385 - DNA, 18 bp, Artificial Sequence
  Probe for the detection of bacteria belonging to the genus Pectinatus
ctgacctgcgattagtag

SEQ ID NO: 386 - DNA, 18 bp, Artificial Sequence
  Probe for the detection of bacteria belonging to the genus Pectinatus
gtgccttgcggcccttag

SEQ ID NO: 387 - DNA, 18 bp, Artificial Sequence
  Probe for the detection of bacteria belonging to the genus Pectinatus
acggtcggtgccttgcgg

SEQ ID NO: 388 - DNA, 18 bp, Artificial Sequence
  Probe for the detection of bacteria belonging to the genus Pectinatus
tactgccattcgtcccct

SEQ ID NO: 389 - DNA, 18 bp, Artificial Sequence
  Probe for the detection of bacteria belonging to the genus Pectinatus
gaccagttcgaatcccat

SEQ ID NO: 390 - DNA, 18 bp, Artificial Sequence
  Probe for the detection of bacteria belonging to the genus Pectinatus
cctcagttcggaccccat

SEQ ID NO: 391 - DNA, 18 bp, Artificial Sequence
  Probe for the detection of bacteria belonging to the genus Pectinatus
actgccattcgtcccctg

SEQ ID NO: 392 - DNA, 18 bp, Artificial Sequence
  Probe for the detection of bacteria belonging to the genus Pectinatus
ttcggaccccatcacggg

SEQ ID NO: 393 - DNA, 18 bp, Artificial Sequence
  Probe for the detection of bacteria belonging to the genus Pectinatus
gttcggaccccatcacgg

SEQ ID NO: 394 - DNA, 18 bp, Artificial Sequence
  Probe for the detection of bacteria belonging to the genus Pectinatus
agttcgaatcccatcacg

SEQ ID NO: 395 - DNA, 18 bp, Artificial Sequence
  Probe for the detection of bacteria belonging to the genus Pectinatus
accagttcgaatcccatc

SEQ ID NO: 396 - DNA, 18 bp, Artificial Sequence
  Probe for the detection of bacteria belonging to the genus Pectinatus
ctcagttcggaccccatc

SEQ ID NO: 397 - DNA, 18 bp, Artificial Sequence
  Probe for the detection of bacteria belonging to the genus Pectinatus
ctgccattcgtcccctgc

SEQ ID NO: 398 - DNA, 18 bp, Artificial Sequence
  Probe for the detection of bacteria belonging to the genus Pectinatus
atccgcttaatgttccgc

SEQ ID NO: 399 - DNA, 18 bp, Artificial Sequence
  Probe for the detection of bacteria belonging to the genus Pectinatus
aagcgacagctaaaagcc

SEQ ID NO: 400 - DNA, 18 bp, Artificial Sequence
  Probe for the detection of bacteria belonging to the genus Pectinatus
atgaccagttcgaatccc

SEQ ID NO: 401 - DNA, 18 bp, Artificial Sequence
  Probe for the detection of Megasphaera cerevisiae
tccaggatcggctccttt

SEQ ID NO: 402 - DNA, 18 bp, Artificial Sequence
  Probe for the detection of Megasphaera cerevisiae
ctccaggatcggctcctt

SEQ ID NO: 403 - DNA, 18 bp, Artificial Sequence
  Probe for the detection of Megasphaera cerevisiae
tcagacgcaaacccctct

SEQ ID NO: 404 - DNA, 18 bp, Artificial Sequence
  Probe for the detection of Megasphaera cerevisiae
gctccaggatcggctcct

SEQ ID NO: 405 - DNA, 18 bp, Artificial Sequence
  Probe for the detection of Megasphaera cerevisiae
ctcttccggcgatagact

SEQ ID NO: 406 - DNA, 18 bp, Artificial Sequence
  Probe for the detection of Megasphaera cerevisiae
gcggcctttagatcgtat

SEQ ID NO: 407 - DNA, 18 bp, Artificial Sequence
  Probe for the detection of Megasphaera cerevisiae
cttccggcgatagactat

SEQ ID NO: 408 - DNA, 18 bp, Artificial Sequence
  Probe for the detection of Megasphaera cerevisiae
cacggcgtatgggtattg

SEQ ID NO: 409 - DNA, 18 bp, Artificial Sequence
  Probe for the detection of Megasphaera cerevisiae
ggtttgctccaggatcgg

SEQ ID NO: 410 - DNA, 18 bp, Artificial Sequence
  Probe for the detection of Megasphaera cerevisiae
cgcaaacccctcttccgg

SEQ ID NO: 411 - DNA, 18 bp, Artificial Sequence
  Probe for the detection of Megasphaera cerevisiae
gggtttgctccaggatcg

SEQ ID NO: 412 - DNA, 18 bp, Artificial Sequence
  Probe for the detection of Megasphaera cerevisiae
tacggtaccgtcacggcg

SEQ ID NO: 413 - DNA, 18 bp, Artificial Sequence
  Probe for the detection of Megasphaera cerevisiae
acgcaaacccctcttccg

SEQ ID NO: 414 - DNA, 18 bp, Artificial Sequence
  Probe for the detection of Megasphaera cerevisiae
cggcgatagactattcag

SEQ ID NO: 415 - DNA, 18 bp, Artificial Sequence
  Probe for the detection of Megasphaera cerevisiae
gacactccagtccggcag

SEQ ID NO: 416 - DNA, 18 bp, Artificial Sequence
  Probe for the detection of Megasphaera cerevisiae
ccaggatcggctcctttc

SEQ ID NO: 417 - DNA, 18 bp, Artificial Sequence
  Probe for the detection of Megasphaera cerevisiae
agacgcaaacccctcttc

SEQ ID NO: 418 - DNA, 18 bp, Artificial Sequence
  Probe for the detection of Megasphaera cerevisiae
tccggcgatagactattc

SEQ ID NO: 419 - DNA, 18 bp, Artificial Sequence
  Probe for the detection of Megasphaera cerevisiae
atcagacgcaaacccctc

SEQ ID NO: 420 - DNA, 18 bp, Artificial Sequence
  Probe for the detection of Megasphaera cerevisiae
gtttgctccaggatcggc

SEQ ID NO: 421 - DNA, 18 bp, Artificial Sequence
  Probe for the detection of Megasphaera cerevisiae
gcaaacccctcttccggc

SEQ ID NO: 422 - DNA, 18 bp, Artificial Sequence
  Probe for the detection of Megasphaera cerevisiae
ccgacactccagtccggc

SEQ ID NO: 423 - DNA, 18 bp, Artificial Sequence
  Probe for the detection of Megasphaera cerevisiae
cctcttccggcgatagac

SEQ ID NO: 424 - DNA, 18 bp, Artificial Sequence
  Probe for the detection of Megasphaera cerevisiae
tcttccggcgatagacta

SEQ ID NO: 425 - DNA, 18 bp, Artificial Sequence
  Probe for the detection of Megasphaera cerevisiae
acggcgtatgggtattga

SEQ ID NO: 426 - DNA, 18 bp, Artificial Sequence
  Probe for the detection of Megasphaera cerevisiae
ccggcgatagactattca

SEQ ID NO: 427 - DNA, 18 bp, Artificial Sequence
  Probe for the detection of Megasphaera cerevisiae
cgacactccagtccggca

SEQ ID NO: 428 - DNA, 18 bp, Artificial Sequence
  Probe for the detection of Megasphaera cerevisiae
tccggcagtttcaatccc

SEQ ID NO: 429 - DNA, 18 bp, Artificial Sequence
  Probe for the detection of Megasphaera cerevisiae
tgctccaggatcggctcc

SEQ ID NO: 430 - DNA, 18 bp, Artificial Sequence
  Probe for the detection of Megasphaera cerevisiae
atgcggcctttagatcgt

SEQ ID NO: 431 - DNA, 18 bp, Artificial Sequence
  Probe for the detection of Megasphaera cerevisiae
atccctggcactcaatgt

SEQ ID NO: 432 - DNA, 18 bp, Artificial Sequence
  Probe for the detection of Megasphaera cerevisiae
aatcagacgcaaacccct

SEQ ID NO: 433 - DNA, 18 bp, Artificial Sequence
  Probe for the detection of Megasphaera cerevisiae
caaacccctcttccggcg

SEQ ID NO: 434 - DNA, 18 bp, Artificial Sequence
  Probe for the detection of Megasphaera cerevisiae
tcatgcggcctttagatc

SEQ ID NO: 435 - DNA, 18 bp, Artificial Sequence
  Probe for the detection of Megasphaera cerevisiae
gacgcaaacccctcttcc

SEQ ID NO: 436 - DNA, 18 bp, Artificial Sequence
  Probe for the detection of Megasphaera cerevisiae
tgcggcctttagatcgta

SEQ ID NO: 437 - DNA, 18 bp, Artificial Sequence
  Probe for the detection of Megasphaera cerevisiae
tctctatccctggcactc

SEQ ID NO: 438 - DNA, 18 bp, Artificial Sequence
  Probe for the detection of Megasphaera cerevisiae
ggctcctttcgcttccct

SEQ ID NO: 439 - DNA, 18 bp, Artificial Sequence
  Probe for the detection of Megasphaera cerevisiae
caggatcggctcctttcg

SEQ ID NO: 440 - DNA, 18 bp, Artificial Sequence
  Probe for the detection of the genus Pectinatus
ccgcacttttaagatccg

SEQ ID NO: 441 - DNA, 18 bp, Artificial Sequence
  Probe for the detection of Pectinatus cerevisiiphilus
gatccgcttagtcatccg

SEQ ID NO: 442 - DNA, 18 bp, Artificial Sequence
  Probe for the detection of Pectinatus cerevisiiphilus
ctactgccattcgtcccc